(19) United States
(12) Patent Application Publication
(10) Pub. No.: US 2026/0226409 A1
(43) Pub. Date: Aug. 6, 2026

(54) METHODS OF PRODUCING GAMMA DELTA T CELLS

(71) Applicant: ANSUN BIOPHARMA, INC, San Diego, CA (US)

(72) Inventors: Wei LIANG, San Diego, CA (US); George WANG, San Diego, CA (US); Nancy CHANG, Houston, TX (US)

(21) Appl. No.: 19/147,955

(22) PCT Filed: Jan. 16, 2024
(86) PCT No.: PCT/US2024/011603

§ 371 Date: Jul. 14, 2025

Related U.S. Application Data
(60) Provisional application No. 63/439,556, filed on Jan. 17, 2023.

(51) Int. Cl. C12N 5/0783; A61K 35/17; A61K 40/11; A61K 40/31; A61K 40/42; A61P 35/00; C07K 14/705; C07K 16/28; C12N 5/00

(52) U.S. Cl. CPC C12N 5/0638; A61K 35/17; A61K 40/11; A61K 40/31; A61K 40/42; A61P 35/00; C07K 14/70503; C07K 16/2809; C12N 5/0087; C12N 5/562; C12N 2500/25; C12N 2501/2315; C12N 2501/33; C12N 2510/00

(57) ABSTRACT

The present application is directed to methods for producing a population of cells comprising y5 T cells. The present application is also directed to, e.g, compositions FIG. 1B useful for such production methods, populations of produced cells, and methods of use thereof.

### CROSS-REFERENCE TO RELATED APPLICATIONS

This application claims the benefit of, and priority to, U.S. Provisional Application 63/439,556, filed on Jan. 17, 2023, the contents of which are hereby incorporated herein by reference in their entirety.

### TECHNICAL FIELD

The present application is directed to methods for producing a population of cells comprising γδ T cells. The present application is also directed to, e.g., compositions and methods useful for such production methods, populations of produced cells, and methods of use thereof.

### BACKGROUND

Adoptive immune cell therapy involves isolating and expanding human immune cells, with or without cell engineering, and administrating these cells to patients for treatment of cancer and other diseases. One promising form of adoptive immune cell therapy is CAR (chimeric antigen receptor) T cell therapy, in which αβ T cells are engineered to express a CAR molecule that recognizes a tumor-associated antigen (TAA) expressed on cancer cell surface, consequently become activated and kill the TAA-expressing cancer cells. Several CAR T cell therapies have been approved for treating hematological malignancies while others are being developed for solid tumors.

To avoid graft-versus-host disease (GVHD) caused by mismatched αβ T cells, the currently approved CAR-T therapies are all autologous cell therapies, meaning that patients' own T cells are used to generate CAR-T cells. Limitations exist for autologous cell therapies, including not able to obtain sufficient number of high-quality T cells from heavily-pretreated cancer patients, the long turnover time (e.g., weeks) for manufacturing CAR-T cells, and the risk of failure in CAR-T production from certain patients. Because of these limitations, allogeneic cell therapies that can be prepared using a variety of sources, such as healthy donor cells, cells from a stem cell bank, or cell lines, and used as off-the-shelf therapies are highly desirable.

Innate immune cell therapies may overcome some of these limitations. Innate immune cells (e.g., NK, NKT, γδ T, and myeloid cells) lack HLA restriction and can regulate allo-responses, thereby avoiding GVHD when adoptively transferring allogeneic innate immune cells (see, e.g., Khatwani et al. Front Immunol, 13, 2022). In particular, NK cells are easily sourced from peripheral blood, present at significantly higher levels than γδ T cells, and easier to generate than γδ T cells. As a result, the majority of innate immune cell therapy innovation focuses on NK cell therapies.

γδ T cells are innate-like T lymphocytes and constitute a small portion (0.5%-5%) of cells in human peripheral blood mononuclear cells. In spite of their low cell numbers in healthy individuals, previous research has revealed strong anti-cancer and anti-infection capabilities of these cells. Unlike the conventional αβ T cells, γδ T cells inhibit tumor growth and eliminate pathogen-infected cells in an MHC-unrestricted manner. This feature makes γδ T cells an enticing choice for allogeneic immune cell therapy with minimal risk of GVHD. γδ T cells are characterized by the expression of a unique TCR composed of a gamma chain and a delta chain, in contrast to the alpha and beta chains in αβ T cells. In humans, there are two major subsets of γδ T cells identified by their delta chain. Vδ1 cells reside predominantly in the peripheral tissues while vδ2 cells are more abundant in the peripheral blood. Whereas the antigens recognized by the vδ1 TCR remain largely unknown, vδ2 cells recognize non-peptide phosphoantigens that are intermediates of the mevalonate metabolism pathway.

Despite the growing interest in developing γδ T cell-based cell therapies, obstacles currently exist limiting the realization of such therapies. One key issue is to generate the large cell numbers of pure γδ T cells needed for adoptive cell therapy (e.g., >1×10⁸ cells/dose) from the limited cell numbers in the starting material (e.g., human peripheral blood) while maintaining γδ T cells' function and cytotoxicity. Current techniques use aminobisphosphonates, antibodies, and artificial antigen-presenting cells (feeder cells) to promote yγδ T cell expansion (see, e.g., Dokouhaki et al., Cancer Lett, 297, 2010; Van Acker et al., J Hematol Oncol, 9, 2016; Li et al., J Immunother, 33, 2010; Salot et al., J Immunol Methods, 326, 2007; Kondo et al., Cytotherapy, 10, 2008; Deniger et al., Cancer Res, 20, 2014; Xiao et al., Cytotherapy , 20, 2018; Zhou et al., Cell Mol Immunol, 9, 2012; and Makkouk et al., J Immunother Cancer, 9, 2021.

However, use of certain manufacturing components results in additional manufacturing considerations and complications. For example, use of feeder cells requires additional QA/QC steps for checking feeder cell contents in the final products and raise safety concerns around the possible introduction of any residual feeder cells (e.g., K562 cancer cells) to human patients.

### BRIEF SUMMARY

In some aspects, provided herein is a method for producing a population of cells comprising γδ T cells, the method comprising: culturing an isolated pool of mixed immune cells depleted of αβ T cells, or a derivative thereof, in a culture medium comprising: (a) a basal medium; (b) a cytokine; and (c) one or more of the following: (i) a metabolic polypeptide; (ii) an iron source; (iii) an antioxidant enzyme cofactor; (iv) a lipid precursor; and (v) a carrier protein; and harvesting the population of cells comprising γδ T cells.

In some embodiments, the population of cells comprising γδ T cells has a γδ T cell purity of at least about 85%. In some embodiments, the population of cells comprising γδ T cells has a γδ T cell purity of at least about 95%.

In some embodiments, the population of cells comprising γδ T cells comprises vδ2 γδ T cells, and, optionally, vδ1 γδ T cells.

In some embodiments, the population of cells comprising γδ T cells has a vδ2 γδ T cell purity of at least about 70% relative to total T cells, such as γδ T cells, in the population. In some embodiments, the population of cells comprising γδ T cells has a vδ2 γδ T cell purity of at least about 95% relative to total T cells, such as γδ T cells, in the population.

In some embodiments, the population of cells comprising γδ T cells comprises less than about 15% of NK cells. In some embodiments, the population of cells comprising γδ T cells comprises less than about 2.5% of NK cells.

In some embodiments, the basal culture medium is NK MACS medium.

In some embodiments, the cytokine is one or more of IL-15, IL-2, IL-7, SCF, TPO, FLT-3L, BMP4, VEGF, or bFGF. In some embodiments, the cytokine is IL-15. In some embodiments, the cytokine is added to the culture medium in an amount of about 100 pg/mL to about 100 ng/mL.

In some embodiments, the metabolic polypeptide is an insulin or analog thereof. In some embodiments, the insulin is a human insulin. In some embodiments, the metabolic polypeptide is added to the culture medium in an amount of about 1 mg/L to about 50 mg/L.

In some embodiments, the iron source is a transferrin. In some embodiments, the transferrin is a human transferrin. In some embodiments, the iron source is added to the culture medium in an amount of about 1 mg/L to about 50 mg/L.

In some embodiments, the antioxidant enzyme cofactor is a selenium compound. In some embodiments, the selenium compound is sodium selenite. In some embodiments, the antioxidant enzyme cofactor is added to the culture medium in an amount of about 0.001 mg/L to about 0.1 mg/L.

In some embodiments, the lipid precursor is ethanolamine. In some embodiments, the lipid precursor is added to the culture medium in an amount of about 1 mg/L to about 10 mg/L.

In some embodiments, the carrier protein is an albumin. In some embodiments, the albumin is a human albumin. In some embodiments, the human albumin is human serum albumin. In some embodiments, the carrier protein is added to the culture medium in an amount of about 100 mg/L to about 5,000 mg/L.

In some embodiments, the culture medium comprises two or more of: (i) the metabolic polypeptide; (ii) the iron source; (iii) the antioxidant enzyme cofactor; (iv) the lipid precursor; and (v) the carrier protein.

In some embodiments, the culture medium comprises three or more of: (i) the metabolic polypeptide; (ii) the iron source; (iii) the antioxidant enzyme cofactor; (iv) the lipid precursor; and (v) the carrier protein.

In some embodiments, the culture medium comprises four or more of: (i) the metabolic polypeptide; (ii) the iron source; (iii) the antioxidant enzyme cofactor; (iv) the lipid precursor; and (v) the carrier protein.

In some embodiments, the culture medium comprises: (i) the metabolic polypeptide; (ii) the iron source; (iii) the antioxidant enzyme cofactor; (iv) the lipid precursor; and (v) the carrier protein.

In some embodiments, the culture medium comprises ITSEA.

In some embodiments, the culture medium further comprises a serum. In some embodiments, the serum is an AB serotype (AB) serum. In some embodiments, the AB serum is a human AB serum. In some embodiments, the serum is added to the culture medium in an amount of about 0.1% (v/v) to about 10% (v/v).

In some embodiments, the culturing is from about 0 to about 14 days.

In some embodiments, the method further comprises cryopreserving the harvested population of cells comprising γδ T cells.

In some embodiments, the isolated pool of mixed immune cells depleted of αβ T cells are derived from Peripheral Blood Mononuclear Cells (PBMCs). In some embodiments, the isolated pool of mixed immune cells depleted of αβ T cells are derived from a stem cell bank or cell line.

In some embodiments, the method further comprises performing a step of αβ T cell depletion on the isolated pool of mixed immune cells. In some embodiments, the step of αβ T cell depletion comprises an antibody-based depletion. In some embodiments, the step of αβ T cell depletion comprises (a) subjecting the isolated pool of mixed immune cells to an anti-TCR α/β antibody; and (b) separating the antibody-conjugated αβ TCR+ T cells from other cells in the isolated pool of mixed immune cells to obtain an αβ T cell depleted population of cells. In some embodiments, the separating is performed by a cell sorting technique.

In some embodiments, the method further comprises cryopreserving cells obtained from the step of 60 β T cell depletion on the isolated pool of mixed immune cells prior to performing the culturing isolated pool of mixed immune cells, or the derivative thereof, in the culture medium.

In some embodiments, the method further comprises isolating the pool of mixed immune cells from a donor. In some embodiments, the donor is not an intended recipient. In some embodiments, the donor is an intended recipient. In some embodiments, the isolating comprising leukapheresis. In some embodiments, the isolating is performed on blood from the donor. In some embodiments, the donor is a human individual. In some embodiments, the method further comprises obtaining blood from the donor.

In some embodiments, the method further comprises performing a step of γδ T cell transduction. In some embodiments, the culturing step is split by a step of transducing the cells in culture to obtain transduced γδ T cells. In some embodiments, the culturing step comprises a first culturing step of about 0 to about 4 days, with the transducing performed on about day 4. In some embodiments, the culturing step comprises a second culturing step of about 0 to about 10 days. In some embodiments, the transducing comprises introducing one or more transgenes. In some embodiments, the one or more transgenes comprises a CAR. In some embodiments, the CAR specifically recognizes globohexaosylceramide, AKR1C3, SSEA-4, or TROP-2.

In some embodiments, the method further comprises cryopreserving the produced population of cells comprising γδ T cells following the harvesting step.

In some embodiments, the method is a feeder cell-free method for producing the population of cells comprising γδ T cells.

In some embodiments, the method is an antibody-free method for producing the population of cells comprising γδ T cells. For example, in some embodiments, the method does not comprise the use of an antibody in culturing the pool of mixed immune cells, or the derivative thereof, after αβ T cell depletion. In some embodiments, the method is a γδ TCR stimulating antibody-free method for producing the population of cells comprising γδ T cells.

In some embodiments, the method does not comprise the use of an aminobisphosphonate in culturing the pool of mixed immune cells, or the derivative thereof. In some embodiments, the aminobisphosphonate is zoledronate or pamidronate, or a mixture thereof.

In other aspects, provided herein is a population of cells comprising γδ T cells produced using any method described herein. In some embodiments, the population comprises at least about 1×10⁸ γδ T cells. In some embodiments, the population comprises one or more transgenes. In some embodiments, the one or more transgenes comprises a CAR.

In other aspects, provided herein is a method of treating a disease in an individual, the method comprising: obtaining an isolated pool of mixed immune cells depleted of αβ T cells, or a derivative thereof, from a donor individual; producing a population of cells comprising γδ T cells using any method described herein; and administering the population of cells comprising γδ T cells to the individual. In some embodiments, the disease is a cancer. In some embodiments, the donor individual is not the individual. In some embodiments, the donor individual is the individual.

It will also be understood by those skilled in the art that changes in the form and details of the implementations described herein may be made without departing from the scope of this disclosure. In addition, although various advantages, aspects, and objects have been described with reference to various implementations, the scope of this disclosure should not be limited by reference to such advantages, aspects, and objects.

All references cited herein, including patent applications and publications, are incorporated herein by reference in their entirety.

### BRIEF DESCRIPTION OF THE DRAWINGS

FIG. 1A the percent (%) of viable γδ T cells after cell expansion (n=4). FIG. 1B provides the percent of yγδ T cell lineages, i.e., vδ1, vδ2, and vδ1-/vδ2-γδ T cells, in the final cell population at Day 14 (n=2). In brief, healthy human peripheral blood apheresis cells were initially depleted of αβ T cells followed by in vitro expansion for 14 days. Cell viabilities after expansion were assessed by the AOPI staining assay. The lineage of γδ T cells was determined by FACS.

FIG. 2A shows the degree of γδ T cell expansion with IL-15 alone, ITSEA alone, and IL-15 and ITSEA in combination. FIG. 2B shows the degree of NK cell expansion under the indicated conditions. FIG. 2C shows the degree of non-γδ T cell/non-NK cell expansion under the indicated conditions.

FIG. 3A shows the percent of γδ T cell purity in the final cell population at Day 14. FIG. 3B shows the percent of NK cell purity in the final cell population at Day 14. FIG. 3C shows the percent of non-γδ T cell/non-NK cell purity in the final cell population at Day 14.

FIG. 4A shows the percent (%) of specific lysis of SKOV3 cancer cells by γδ T cells that were expanded in vitro for 14 days. FIG. 4B shows the percent (%) of specific lysis of A549 cancer cells by γδ T cells that were expanded in vitro for 14 days. In brief, luciferase-expressing tumor cells (i.e., SKOV3 and A549) that were pretreated with or without zoledronate (Zol) were co-cultured with γδ T cells at E/T=4/1 in the presence of rhIL-15 in culture. Cytotoxicity (Specific lysis %) was determined at Day 1 and Day 3 based on the loss of the luciferase signals (n=3 technical replicates). rhIL-15: recombinant human IL-15; Zol: zolendronate; E/T: Effector cell to Target cell ratio.

FIG. 5A shows the percent (%) of specific lysis of SKOV3 cancer cells grown in 3D model by γδ T cells that were expanded in vitro for 14 days. FIG. 5B shows the percent (%) of specific lysis of A549 cancer cells grown in 3D model by γδ T cells that were expanded in vitro for 14 days. In brief, fluorescent-tagged tumor cells were plated in 3D culture to allow tumorsphere formation. Two days later, γδ T cells were added at E/T: 4/1 with rhIL-15. Fluorescent intensities signifying tumor mass were determined 3 and 6 days later (n=3 technical replicates). TC: tumor cell; rhIL-15: recombinant human IL-15; E/T: Effector cell to Target cell ratio.

FIG. 6A provides a schematic diagram of the anti-TROP-2 CAR construct to be transduced into the αβ T cell-depleted cells prior to further expansion. FIG. 6B shows the transduction efficiency of the non-transduced and the anti-TROP-2 CAR-transduced γδ T cells. FIG. 6C shows the fold expansion of the non-transduced and the anti-TROP-2 CAR-transduced γδ T cells. FIG. 6D shows the increased antitumor cytotoxic activity of the γδ CAR-T cells against a TROP-2-expressing cell line (i.e., SKOV3), whereas FIG. 6E shows no significant change above non-transduced γδ T cells against a cell line that does not express TROP-2 (i.e., A549). n=3 technical replicates per condition.

### DETAILED DESCRIPTION

The present application provides, in some aspects, methods of producing a population of cells comprising γδ T cells, the methods comprising culturing using a culture medium described herein. In other aspects, provided herein are compositions useful for such production methods, populations of produced cells, and methods of use thereof. The disclosure of the present application is based on the inventors' unique perspectives and findings regarding a method for rapid expansion of γδ T cells in vitro using a novel feeder cell-free and an antibody-free platform that selectively and robustly expands unmodified or genetically engineered human γδ T cells. In certain embodiments, the resulting γδ T cells are mainly composed of the vδ2 subtype of γδ T cells and show potent antitumor activities. In certain embodiments, the disclosure provided herein eliminates the extra manufacturing steps for feeder cells and antibodies, and thus the QA/QC steps for checking feeder cell contents in the final products, and the safety concerns of introducing residual feeder cells (e.g., cancer cells) to humans, such as human patients. As demonstrated therein, the produced cell populations comprising γδ T cells have a sufficient purity so as to not require any additional enrichment or isolation after cell expansion and to minimize risk for Graft versus Host Disease (GvHD) upon administration to patients as an allogeneic cell therapy, due to the lack of αβ T cells. Thus, as described in further detail herein, the populations of cell comprising γδ T cells obtained by the methods taught herein can be used safely, efficiently, and effectively in prophylactic, therapeutic, experimental, and commercial applications.

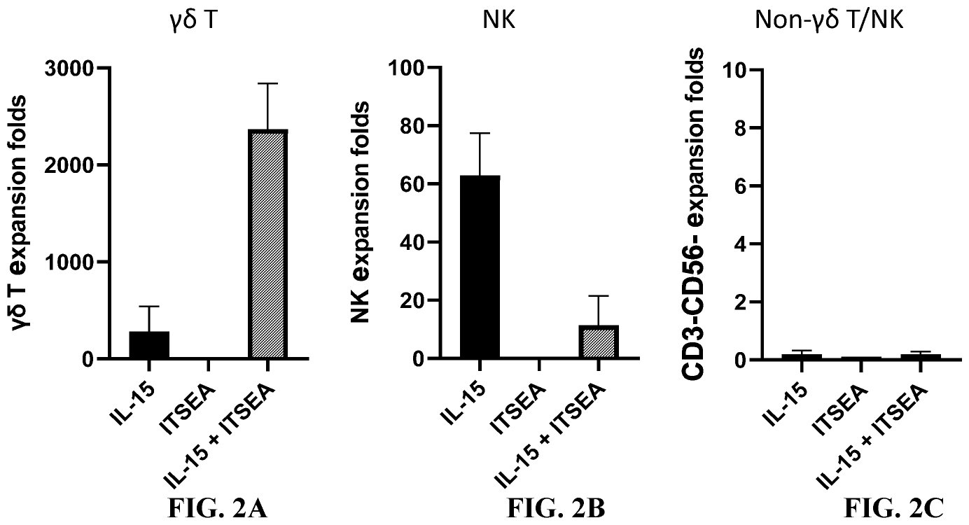

More specifically, e.g., after extensive investigation, the methods described herein for expanding γδ T cells in vitro were unexpectedly found to generate populations of cells comprising γδ T cells that had expanded γδ T cells at significant levels, as compared to (e.g.) feeder cell-free methods. In such produced populations of cells comprising γδ T cells, it was also found that NK cells were not significantly expanded and remained at low levels, such as less than 5% of cells of the population. For example, the method described in Example 1 below generated a >2000-fold expansion of γδ T cells on average, as shown in FIG. 2A. The robustness of the γδ T cell expansion and purity of the expansion methods described herein was unexpected in light of the prevalence in the art of methods using, e.g., feeder cells and/or γδ T cell-activating antibodies to drive γδ T cell expansion without simultaneously promoting NK cell expansion. Thus, the novel methods described herein provide similar or better efficacy while also providing a safer product using a more efficient protocol than currently described protocols in the art.

Thus, in some aspects, provided herein is a method for producing a population of cells comprising γδ T cells, the method comprising: culturing an isolated pool of mixed immune cells depleted of αβ T cells, or a derivative thereof, in a culture medium comprising (a) a basal medium; (b) a cytokine; and (c) one or more of the following: (i) a metabolic polypeptide; (ii) an iron source; (iii) an antioxidant enzyme cofactor; (iv) a lipid precursor; and (v) a carrier protein; and, optionally, harvesting the population of cells comprising γδ T cells. In some embodiments, the basal medium is the NK MACS medium (Miltenyi Biotec). In some embodiments, the αβ T cell-depleted pool of mixed immune cells are isolated peripheral blood mononuclear cells (PBMCs) after αβ T cell depletion treatment. In some embodiments, the population of cells comprises γδ T cells having a γδ T cell purity of at least about 95%. In some embodiments, the population of cells comprises γδ T cells comprising vδ1 γδ T cells and vδ2 γδ T cells. In some embodiments, the population of cells comprising γδ T cells comprises less than about 5% NK cells. In some embodiments, the cytokine is IL-15. In some embodiments, the metabolic polypeptide is an insulin or analog thereof. In some embodiments, the iron source is a transferrin. In some embodiments, the antioxidant enzyme cofactor is a selenium compound (such as sodium selenite). In some embodiments, the lipid precursor is ethanolamine. In some embodiments, the carrier protein is an albumin (such as human albumin, including human serum albumin). In some embodiments, the culturing is from about 0 to about 14 days. In some embodiments, the method is a feeder cell-free method for producing the population of cells comprising γδ T cells. In some embodiments, the method is an antibody-free method for producing the population of cells comprising γδ T cells. For example, in some embodiments, the method does not comprise the use of an antibody in culturing the pool of mixed immune cells, or the derivative thereof, after αβ T cell depletion. In some embodiments, the method is a γδ TCR-stimulating antibody-free method for producing the population of cells comprising γδ T cells. In some embodiments, the method does not comprise the use of an aminobisphosphonate in culturing the PBMCs, or a derivative thereof.

In other aspects, provided herein is a method for producing a population of cells comprising γδ T cells, wherein the γδ T cells are engineered to comprise a transgene, the method comprising: culturing an isolated pool of mixed immune cells depleted of αβ T cells, or a derivative thereof, in a culture medium comprising: (a) a basal medium; (b) a cytokine; and (c) one or more of the following: (i) a metabolic polypeptide; (ii) an iron source; (iii) an antioxidant enzyme cofactor; (iv) a lipid precursor; and (v) a carrier protein; transducing the population of cells comprising the γδ T cells (or an intermediate/precursor thereof) to deliver a transgene to the γδ T cells; and, optionally, harvesting the population of cells comprising γδ T cells. In some embodiments, the method further comprises performing a step of αβ T cell depletion (such as antibody-based depletion) on the isolated pool of mixed immune cells depleted of αβ T cells. In some embodiments, the population of cells comprises γδ T cells having a γδ T cell purity of at least about 95%. In some embodiments, the population of cells comprises γδ T cells comprising mainly vδ2 γδ T cells, and, optionally, vδ1 γδ T cells. In some embodiments, the population of cells comprising γδ T cells comprises less than about 5% NK cells. In some embodiments, the cytokine is IL-15. In some embodiments, the metabolic polypeptide is an insulin or analog thereof. In some embodiments, the iron source is a transferrin. In some embodiments, the antioxidant enzyme cofactor is a selenium compound (such as sodium selenite). In some embodiments, the lipid precursor is ethanolamine. In some embodiments, the carrier protein is an albumin (such as human albumin, including human serum albumin). In some embodiments, the transgene is a CAR. In some embodiments, the culturing is from about 0 to about 14 days. In some embodiments, the method is a feeder cell-free method for producing the population of cells comprising γδ T cells. In some embodiments, the method is an antibody-free method for producing the population of cells comprising γδ T cells. For example, in some embodiments, the method does not comprise the use of an antibody in culturing the pool of mixed immune cells, or the derivative thereof, after αβ T cell depletion. In some embodiments, the method is a γδ TCR-stimulating antibody-free method for producing the population of cells comprising γδ T cells. In some embodiments, the method does not comprise the use of an aminobisphosphonate in culturing the pool of mixed immune cells depleted of αβ T cells, or the derivative thereof.

In other aspects, provided herein is a population of cells comprising γδ T cells produced using the method as described herein.

In another aspect, provided herein is a method of treating a disease in an individual, the method comprising: obtaining an isolated pool of mixed immune cells depleted of αβ T cells, or a derivative thereof, from a donor individual; producing a population of cells comprising γδ T cells using a method described herein; and administering the population of cells comprising γδ T cells to the individual. In some embodiments, the disease is a cancer. In some embodiments, the donor individual is not the individual administered the population of cells comprising γδ T cells (i.e., the method of treatment is an allogeneic treatment). In some embodiments, the donor individual is the individual administered the population of cells comprising γδ T cells (i.e., the method of treatment is an autologous treatment).

Also provided are kits for manufacturing the population of cells comprising γδ T cells described herein, pharmaceutical compositions and kits comprising the population of cells comprising γδ T cells described herein, and methods of use thereof for treating or otherwise ameliorating diseases, such as cancers.

The section headings used herein are for organizational purposes only and are not to be construed as limiting the subject matter described. For example, some aspects of the disclosure are presented in a modular fashion, and such presentation is not to be construed as limited the possible combinations of approaches taught herein.

### I. Definitions

For purposes of interpreting this specification, the following definitions will apply and, whenever appropriate, terms used in the singular will also include the plural and vice versa. In the event that any definition set forth below conflicts with any document incorporated herein by reference, the definition set forth shall control.

The terms “polypeptide” and “protein,” as used herein, may be used interchangeably to refer to a polymer comprising amino acid residues, and are not limited to a minimum length. Such polymers may contain natural or non-natural amino acid residues, or combinations thereof, and include, but are not limited to, peptides, polypeptides, oligopeptides, dimers, trimers, and multimers of amino acid residues. Full-length polypeptides or proteins, and fragments thereof, are encompassed by this definition. The terms also include modified species thereof, e.g., post-translational modifications of one or more residues, for example, methylation, phosphorylation glycosylation, sialylation, or acetylation.

As used herein, “treatment” or “treating” is an approach for obtaining beneficial or desired results including clinical results. For purposes of this application, beneficial or desired clinical results include, but are not limited to, one or more of the following: decreasing one more symptoms resulting from the disease, diminishing the extent of the disease, stabilizing the disease (e.g., preventing or delaying the worsening of the disease), preventing or delaying the spread of the disease, preventing or delaying the occurrence or recurrence of the disease, delay or slowing the progression of the disease, ameliorating the disease state, providing a remission (whether partial or total) of the disease, decreasing the dose of one or more other medications required to treat the disease, delaying the progression of the disease, increasing the quality of life, and/or prolonging survival. Also encompassed by “treatment” is a reduction of pathological consequence of the disease. The methods of the present application contemplate any one or more of these aspects of treatment.

The terms “individual,” “subject” and “patient” are used interchangeably herein to describe a mammal, including humans. In some embodiments, the individual is human. In some embodiments, an individual suffers from a cancer. In some embodiments, the individual is in need of treatment.

The term “antibody” is used in its broadest sense and encompasses various antibody structures, including but not limited to monoclonal antibodies, polyclonal antibodies, multi-specific antibodies (e.g., bispecific antibodies, trispecific antibodies, etc.), humanized antibodies, chimeric antibodies, full-length antibodies and antigen-binding fragments, single chain Fv, nanobodies, Fc fusion proteins, thereof, so long as they exhibit the desired antigen-binding activity. Antibodies and/or antibody fragments may be derived from murine antibodies, rabbit antibodies, chicken antibodies, human antibodies, fully humanized antibodies, camelid antibody variable domains and humanized versions, shark antibody variable domains and humanized versions, and camelized antibody variable domains.

It will be understood by one of ordinary skill in the art that the term “cell” includes the primary subject cell and its progeny.

A “pharmaceutically acceptable carrier” refers to one or more ingredients in a pharmaceutical formulation, other than an active ingredient, which is nontoxic to a subject. A pharmaceutically acceptable carrier includes, but is not limited to, a buffer, excipient, stabilizer, cryoprotectant, tonicity agent, preservative, and combinations thereof. Pharmaceutically acceptable carriers or excipients have preferably met the required standards of toxicological and manufacturing testing and/or are included on the Inactive Ingredient Guide prepared by the U.S. Food and Drug administration or other state/federal government or listed in the U.S. Pharmacopeia or other generally recognized pharmacopeia for use in mammals, and more particularly in humans.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

### II. Methods of Producing a Population of Cells Comprising γδ T Cells

Provided herein, in some aspects, are methods of producing a population of cells comprising γδ T cells involving the culturing populations of PBMCs, or derivatives thereof. In some embodiments, the methods comprise steps associated with cell depletion (e.g., αβ T cell depletion), obtaining an isolated pool of mixed immune cells, such as a population comprising PBMCs, transduction, and cryopreservation. In some embodiments, the culturing comprises use of a culture medium comprising: (a) a basal medium; (b) a cytokine (e.g., IL-15); and (c) one or more (including all) of the following: (i) a metabolic polypeptide (e.g., an insulin); (ii) an iron source (e.g., a transferrin); (iii) an antioxidant enzyme cofactor (e.g., a selenium compound); (iv) a lipid precursor (e.g., ethanolamine); and (v) a carrier protein (e.g., an albumin). In some embodiments, the method is a feeder cell-free method for producing the population of cells comprising γδ T cells. In some embodiments, the method is an antibody-free method (such as a γδ TCR-stimulating antibody-free method) for producing the population of cells comprising γδ T cells. In some embodiments, the method does not comprise the use of an aminobisphosphonate in culturing the isolated pool of mixed immune cells, such as PBMCs, or the derivative thereof.

In some embodiments, the population of cells comprising γδ T cells produced using the methods provided herein has a γδ T cell purity of at least about 85%, such as at least about any of 86%, 87%, 88%, 89%, 90%, 91%, 92%, 93%, 94%, 95%, 95.5%, 96%, 96.5%, 97%, 97.5%, 98%, 98.5%, 99%, 99.5%, or 100%. In some embodiments, the population of cells comprising γδ T cells produced using the methods provided herein comprises vδ2 γδ T cells. In some embodiments, the population of cells comprising γδ T cells produced using the methods provided herein comprises vδ2 γδ T cells and vδ1 γδ T cells. In some embodiments, the population of cells comprising γδ T cells produced using the methods provided herein has a vδ2 γδ T cell purity, such as relative to total T cells, such as total γδ T cells, in the population, of at least about 70%, such as at least about any of 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, or 100%. In some embodiments, the population of cells comprising γδ T cells produced using the methods provided herein comprises less than about 15% of NK cells, such as less than about any of 14% of NK cells, 13% of NK cells, 12% of NK cells, 11% of NK cells, 10% of NK cells, 9% of NK cells, 8% of NK cells, 7% of NK cells, 6% of NK cells, 5% of NK cells, 4.5% of NK cells, 4% of NK cells, 3.5% of NK cells, 3% of NK cells, 2.5% of NK cells, 2% of NK cells, 1.5% of NK cells, 1% of NK cells, or 0.5% of NK cells.

Certain aspects of the methods taught herein are discussed in more detail in a modular fashion below. One of ordinary skill in the art will readily understand how the aspects of the present description can be combined to obtain any method of producing a population of cells comprising γδ T cells encompassed by the teachings provided herein. The discussion of such methods, including components and steps thereof, in a modular fashion does not limit the scope of the description encompassed herein.

### A. Culturing

The methods of producing a population of cells comprising γδ T cells comprise culturing an isolated pool of mixed immune cells depleted of αβ T cells, such as a population of cells comprising PBMCs depleted of αβ T cells, or derivatives thereof, in a culture medium. In some embodiments, as used herein, the isolated pool of mixed immune cells, or a derivative thereof, refers to a population of cells obtained from any source, such as an individual, or a processed population of cells, such as processed via an αβ depletion process, however, derivatives thereof do not include the harvested population of cells comprising γδ T cells produced using the methods described herein.

Many anatomical sources of mixed immune cell populations have been identified in humans, including methods of immune cell harvesting. These include circulating peripheral blood mononuclear cells, resident skin or mucosal immune cells, bone marrow immune cells, immune cells circulating in lymph and the lymph nodes, tissue resident immune cells from an organ biopsy, immune cells in a tumor or tumor microenvironment, immune cells in cerebrospinal fluid (see, e.g., Schröder et al. (2018), Front Neurol 9: 1081), resident immune cells of the peritoneum (see, e.g., Shen et al. (2015), Stem Cell Res 15(2): 299-304; and Ruiz-Alcaraz et al. (2020), Immunol Cell Biol, 98(2): 114-126), etc. In some embodiments, the isolated pool of mixed immune cells depleted of αβ T cells may be obtained from any source known in the art, including but not limited to PBMCs, cell banks (e.g., stem cell bank, blood cell bank, cell line bank), induced pluripotent stem cells, bone marrow aspirate, peritoneal immune isolate (e.g., peritoneal dialysis), immune isolate from lymph and/or lymph node collection, mucosal immune isolate, or skin biopsy immune isolate. In some embodiments, the isolated pool of mixed immune cells depleted of αβ T cells comprises PBMCs depleted of αβ T cells. In some embodiments, the isolated pool of mixed immune cells depleted of αβ T cells is a freshly isolated cell population. In some embodiments, the isolated pool of mixed immune cells depleted of αβ T cells is a cryopreserved cell population.

In some embodiments, the culturing step comprises culturing an isolated pool of mixed immune cells depleted of αβ T cells, or a derivative thereof, in a culture medium for a period of time until harvesting the desired population of cells comprising γδ T cells. In certain aspects of the description provided herein, culturing includes time-based features, such as the time the isolated pool of mixed immune cells depleted of αβ T cells, or a derivative thereof, are cultured in a culture medium. In such embodiments, initial culturing with a culture medium used for γδ T cell expansion, such as described below, can be use at the starting time point, e.g., day 0. In some embodiments, the culturing comprises changing the culture medium, such as remove the existing medium and replacing with fresh culture medium. For example, in some embodiments, the culture medium is changed every 2-10 days, such as every 4-7 days. In some embodiments, the culturing comprises adding a culture medium component, such as a cytokine (e.g., IL-15), without changing the culture medium. For example, in some embodiments, the cytokine of the culture medium is added every 1-4 days, such as every 2-3 days, of the culturing process.

In some embodiments, the culturing proceeds for about 30 days or less, such as about any of 28 days or less, 21 days or less, or 14 days or less. In some embodiments, the culturing period is based on obtaining a resulting population of cells comprising γδ T cells, such as evaluated with one or more markers. In some embodiments, the culturing period is based on obtaining a number of cells in the population of cells and/or a number of γδ T cells in the population of cells. In some embodiments, the culturing period is based on obtaining a fold expansion, such as 500-fold, 1000-fold, 1500-fold, or 2000-fold expansion of γδ T cells in the population of cells comprising γδ T cells as compared to the starting PBMCs, or derivative thereof.

Culturing techniques, including culturing techniques for expanding γδ T cells, are well known in the art. See, e.g., Segeritz et al., Basic Science Methods for Clinical Researchers, 2017; and Fundamental Techniques in Cell Culture, 3rd Edition, 2016, the contents of which are incorporated herein by reference in their entirety. Common culturing conditions often include culturing cells at 37° C., in a standard atmosphere comprising 5% CO₂, where relative humidity is maintained at about 100%. One of ordinary skill of art will readily appreciate that certain conditions, such as temperature, percent CO₂, humidity, oxygen, and pH for culturing can be adjusted to still provide suitable culture conditions for expanding γδ T cells. In certain aspects, such techniques may be configured to produce a product suitable for human use, e.g., administration of a population of cell comprising γδ T cells. In some embodiments, the culturing technique is a good manufacturing process (GMP).

### i. Culture Medium and Components Thereof

Various culture mediums taught herein are useful for the taught methods of producing a population of cells comprising γδ T cells. The culture medium provided herein comprises: (a) a basal medium; (b) a cytokine; and (c) one or more (including all) of the following: (i) a metabolic polypeptide; (ii) an iron source; (iii) an antioxidant enzyme cofactor; (iv) a lipid precursor; and (v) a carrier protein. In some embodiments, the culture medium further comprises a serum. In some embodiments, the method of producing a population of cells comprising γδ T cells uses a single type of culture medium. In some embodiments, two or more different culture media may be used in a method of producing a population of cells comprising γδ T cells, e.g., the method comprises culturing with a first culture medium according to the disclosure provided herein and culturing with a second culture medium according to the disclosure provided herein, wherein the first culture medium and the second culture medium are different.

The culture mediums described herein comprise a basal culture medium. In some embodiments, the basal culture medium is selected from the group consisting of NK MACS, TexMACS, RPMI-1640, AIM-V, OpTmizer, DMEM, IMDM, and X-VIVO. In some embodiments, the basal culture medium comprises, or is, NK MACS medium. In some embodiments, the cytokine is a mitogenic cytokine. In some embodiments, the cytokine is one or more of IL-15, IL-2, IL-7, IL-12, IL-18, IL-21, SCF, TPO, FLT-3L, BMP4, VEGF, or bFGF. In some embodiments, the cytokine is IL-15. In some embodiments, the cytokine is added to the culture medium in an amount of about 100 pg/mL to about 500 ng/ml, such as any of about 1 ng/ml to about 100 ng/mL, or about 1 ng/ml to about 20 ng/mL, or about 5 ng/mL to about 15 ng/ml, or about 5 ng/ml to about 500 ng/ml, or about 1 ng/ml to about 500 ng/mL. In some embodiments, the cytokine is added to the culture medium in an amount of about any of 1 ng/ml, 2 ng/ml, 3 ng/ml, 4 ng/mL, 5 ng/ml, 6 ng/ml, 7 ng/ml, 8 ng/ml, 9 ng/ml, 10 ng/mL, 11 ng/ml, 12 ng/ml, 13 ng/ml, 14 ng/ml, 15 ng/ml, 16 ng/ml, 17 ng/ml, 18 ng/ml, 19 ng/ml, or 20 ng/mL. In some embodiments, the cytokine is added to the culture medium during culturing every 1-4 days, e.g., every 2 days or every 3 days, such as in the amounts described herein for each instance of the cytokine being added to the culture medium.

In some embodiments, the metabolic polypeptide is a hormone, such as a human hormone. In some embodiments, the metabolic polypeptide is selected from the group consisting of an insulin, a proinsulin, and an insulin-like growth factor. In some embodiments, the metabolic polypeptide is an insulin or analog thereof. In some embodiments, the insulin is a human insulin. In some embodiments, the metabolic polypeptide (e.g., insulin) is added to the culture medium in an amount of about 1 mg/L to about 50 mg/L, such as any of about 1 mg/L to about 18 mg/L, or about 15 mg/L to about 25 mg/L, or about 18 mg/L to about 22 mg/L, or about 20 mg/L to about 40 mg/L, or about 35 mg/L to about 50 mg/L. In some embodiments, the metabolic polypeptide (e.g., insulin) is added to the culture medium in an amount of about any of 15 mg/L, 16 mg/L, 17 mg/L, 18 mg/L, 19 mg/L, 20 mg/L, 21 mg/L, 22 mg/L, 23 mg/L, 24 mg/L, or 25 mg/L.

In some embodiments, the iron source increases cellular uptake of non-heme iron, such as via a transferrin or non-transferrin-based cellular mechanism. In some embodiments, the iron source is a transferrin. In some embodiments, the transferrin is a human transferrin. In some embodiments, the iron source is a component of non-transferrin bound iron (NTBI) uptake, such as DCYTB, DMT1, ZIP8, and ZIP14, or increases activity of expression of such one or more components. In some embodiments, the iron source (e.g., transferrin) is added to the culture medium in an amount of about 1 mg/L to about 50 mg/L, such as about 5 mg/L to about 45 mg/L. In some embodiments, the iron source (e.g., transferrin) is added to the culture medium in an amount of about any of 5 mg/L, 6 mg/L, 7 mg/L, 8 mg/L, 9 mg/L, 10 mg/L, 11 mg/L, 12 mg/L, 13 mg/L, 14 mg/L, 15 mg/L, 16 mg/L, 17 mg/L, 18 mg/L, 19 mg/L, 20 mg/L, 25 mg/L, 30 mg/L, 35 mg/L, 40 mg/L, 45 mg/L, or 50 mg/L.

In some embodiments, the antioxidant enzyme cofactor, such as a cofactor for a glutathione peroxidase, is a selenium compound. In some embodiments, the selenium compound is a selenite (a salt comprising selenium). In some embodiments, the selenium compound is sodium selenite, or disodium selenite. In some embodiments, the antioxidant enzyme cofactor (e.g., sodium selenite) is added to the culture medium in an amount of about 0.001 mg/L to about 0.1 mg/L. In some embodiments, the antioxidant enzyme cofactor (e.g., sodium selenite) is added to the culture medium in an amount of about any of 0.001 mg/L, 0.005 mg/L, 0.01 mg/L, 0.05 mg/L, or 0.1 mg/L. In some embodiments, the antioxidant enzyme cofactor (e.g., sodium selenite) is added to the culture medium in an amount of about any 0.01 mg/L, 0.011 mg/L, 0.012 mg/L, 0.013 mg/L, 0.014 mg/L, 0.015 mg/L, 0.016 mg/L, 0.017 mg/L, 0.018 mg/L, 0.019 mg/L, or 0.02 mg/L.

In some embodiments, the lipid precursor is ethanolamine or a glyceride, such as diglyceride. In some embodiments, the lipid precursor is ethanolamine. In some embodiments, the lipid precursor (e.g., ethanolamine) is added to the culture medium in an amount of about 1 mg/L to about 10 mg/L. In some embodiments, the lipid precursor (e.g., ethanolamine) is added to the culture medium in an amount of about any of 1 mg/L, 2 mg/L, 3 mg/L, 4 mg/L, 5 mg/L, 6 mg/L, 7 mg/L, 8 mg/L, 9 mg/L, or 10 mg/L.

In some embodiments, the carrier protein is a globular protein. In some embodiments, the carrier protein is selected from the group consisting of albumin, lysozyme, and myoglobin. In some embodiments, the carrier protein is an albumin. In some embodiments, the albumin is a human albumin. In some embodiments, the human albumin is human serum albumin. In some embodiments, the carrier protein (e.g., albumin) is added to the culture medium in an amount of about 100 mg/L to about 5,000 mg/L. In some embodiments, the carrier protein (e.g., albumin) is added to the culture medium in an amount of about any of 100 mg/L, 200 mg/L, 300 mg/L, 400 mg/L, 500 mg/L, 600 mg/L, 700 mg/L, 800 mg/L, 900 mg/L, 1000 mg/L, 1500 mg/L, 2000 mg/L, 2500 mg/L, 3000 mg/L, 3500 mg/L, 4000 mg/L, 4500 mg/L, or 5000 mg/L.

In some embodiments, the culture medium comprises (such as comprises the following components added to a basal medium, e.g., NK MACS) two or more of, three or more of, four or more of, or all of: (i) the metabolic polypeptide (e.g., insulin); (ii) the iron source (e.g., transferrin); (iii) the antioxidant enzyme cofactor (e.g., a selenium compound); (iv) the lipid precursor (e.g., ethanolamine); or (v) the carrier protein (e.g., albumin). In some embodiments, the culture medium comprises (such as comprises the following component added to a basal medium) ITSEA. In some embodiments, the metabolic polypeptide (e.g., insulin) is added in an amount of about 15 mg/L to about 25 mg/L, such as about any of 16 mg/L, 17 mg/L, 18 mg/L, 19 mg/L, 20 mg/L, 21 mg/L, 22 mg/L, 23 mg/L, or 24 mg/L. In some embodiments, the iron source (e.g., transferrin) is added in an amount of about 5 mg/L to about 15 mg/L, such as about any of 6 mg/L, 7 mg/L, 8 mg/L, 9 mg/L, 10 mg/L, 11 mg/L, 12 mg/L, 13 mg/L, or 14 mg/L. In some embodiments, the antioxidant enzyme cofactor (e.g., a selenium compound) is added in an amount of about 0.01 mg/L to about 0.02 mg/L, such as about any of 0.011 mg/L, 0.012 mg/L, 0.013 mg/L, 0.014 mg/L, 0.015 mg/L, 0.016 mg/L, 0.017 mg/L, 0.018 mg/L, or 0.019 mg/L. In some embodiments, the lipid precursor (e.g., ethanolamine) is added in an amount of about 1 mg/L to about 10 mg/L, such as about any of 2 mg/L, 3 mg/L, 4 mg/L, 5 mg/L, 6 mg/L, 7 mg/L, 8 mg/L, or 9 mg/L. In some embodiments, the carrier protein (e.g., albumin) is added in an amount of about 100 mg/L to about 1000 mg/L, such as about any of 200 mg/L, 300 mg/L, 400 mg/L, 500 mg/L, 600 mg/L, 700 mg/L, 800 mg/L, or 900 mg/L.

In some embodiments, the culture medium comprises (such as comprises the following components added to a basal medium, e.g., NK MACS) a cytokine (e.g., IL-15) and two or more of, three or more of, four or more of, or all of: (i) the metabolic polypeptide (e.g., insulin); (ii) the iron source (e.g., transferrin); (iii) the antioxidant enzyme cofactor (e.g., a selenium compound); (iv) the lipid precursor (e.g., ethanolamine); or (v) the carrier protein (e.g., albumin). In some embodiments, the culture medium comprises (such as comprises the following components added to a basal medium) a cytokine (e.g., IL-15) and ITSEA. In some embodiments, the cytokine (e.g., IL-15) is added in an amount of about 1 ng/ml to about 500 ng/ml, such as about any of 2 ng/ml, 3 ng/mL, 4 ng/ml, 5 ng/mL, 6 ng/ml, 7 ng/mL, 8 ng/mL, 9 ng/mL, 10 ng/ml, 11 ng/ml, 12 ng/mL, 13 ng/ml, 14 ng/ml, 15 ng/ml, 16 ng/ml, 17 ng/ml, 18 ng/ml, or 19 ng/mL. In some embodiments, the cytokine is added to the culture medium during culturing every 1-4 days, e.g., every 2 days or every 3 days, such as in the amounts described herein for each instance of the cytokine being added to the culture medium. In some embodiments, the metabolic polypeptide (e.g., insulin) is added in an amount of about 15 mg/L to about 25 mg/L, such as about any of 16 mg/L, 17 mg/L, 18 mg/L, 19 mg/L, 20 mg/L, 21 mg/L, 22 mg/L, 23 mg/L, or 24 mg/L. In some embodiments, the iron source (e.g., transferrin) is added in an amount of about 5 mg/L to about 15 mg/L, such as about any of 6 mg/L, 7 mg/L, 8 mg/L, 9 mg/L, 10 mg/L, 11 mg/L, 12 mg/L, 13 mg/L, or 14 mg/L. In some embodiments, the antioxidant enzyme cofactor (e.g., a selenium compound) is added in an amount of about 0.01 mg/L to about 0.02 mg/L, such as about any of 0.011 mg/L, 0.012 mg/L, 0.013 mg/L, 0.014 mg/L, 0.015 mg/L, 0.016 mg/L, 0.017 mg/L, 0.018 mg/L, or 0.019 mg/L. In some embodiments, the lipid precursor (e.g., ethanolamine) is added in an amount of about 1 mg/L to about 10 mg/L, such as about any of 2 mg/L, 3 mg/L, 4 mg/L, 5 mg/L, 6 mg/L, 7 mg/L, 8 mg/L, or 9 mg/L. In some embodiments, the carrier protein (e.g., albumin) is added in an amount of about 100 mg/L to about 1000 mg/L, such as about any of 200 mg/L, 300 mg/L, 400 mg/L, 500 mg/L, 600 mg/L, 700 mg/L, 800 mg/L, or 900 mg/L.

In some embodiments, the culture medium further comprises a serum. In some embodiments, the serum is an AB serotype (AB) serum. In some embodiments, the serum is a human AB serum. In some embodiments, the human AB serum is collected from a human male with AB blood type. In some embodiments, the human AB serum has reduced reactivity against the γδ T cells being grown in the culture medium. In some embodiments, the serum is added to the culture medium in an amount of about 0.1% (v/v) to about 10% (v/v), such as about any of 0.5% (v/v), 1% (v/v), 1.5% (v/v), 2% (v/v), 2.5% (v/v), 3% (v/v), 3.5% (v/v), 4% (v/v), 4.5% (v/v), 5% (v/v), 5.5% (v/v), 6% (v/v), 6.5% (v/v), 7% (v/v), 7.5% (v/v), 8% (v/v), 8.5% (v/v), 9% (v/v), 9.5% (v/v), or 10% (v/v).

In some embodiments, the culture medium comprises (such as comprises the following components added to a basal medium, e.g., NK MACS) a cytokine (e.g., IL-15), a serum (e.g., AB serum), and two or more of, three or more of, four or more of, or all of: (i) the metabolic polypeptide (e.g., insulin); (ii) the iron source (e.g., transferrin); (iii) the antioxidant enzyme cofactor (e.g., a selenium compound); (iv) the lipid precursor (e.g., ethanolamine); or (v) the carrier protein (e.g., albumin). In some embodiments, the culture medium comprises (such as comprises the following components added to a basal medium) a cytokine (e.g., IL-15) and ITSEA. In some embodiments, the cytokine (e.g., IL-15) is added in an amount of about 1 ng/ml to about 50 ng/mL, such as about any of 2 ng/ml, 3 ng/ml, 4 ng/ml, 5 ng/mL, 6 ng/ml, 7 ng/ml, 8 ng/ml, 9 ng/ml, 10 ng/ml, 11 ng/ml, 12 ng/ml, 13 ng/ml, 14 ng/ml, 15 ng/ml, 16 ng/ml, 17 ng/ml, 18 ng/ml, or 19 ng/mL. In some embodiments, the cytokine is added to the culture medium during culturing every 1-4 days, e.g., every 2 days or every 3 days, such as in the amounts described herein for each instance of the cytokine being added to the culture medium. In some embodiments, the metabolic polypeptide (e.g., insulin) is added in an amount of about 15 mg/L to about 25 mg/L, such as about any of 16 mg/L, 17 mg/L, 18 mg/L, 19 mg/L, 20 mg/L, 21 mg/L, 22 mg/L, 23 mg/L, or 24 mg/L. In some embodiments, the iron source (e.g., transferrin) is added in an amount of about 5 mg/L to about 15 mg/L, such as about any of 6 mg/L, 7 mg/L, 8 mg/L, 9 mg/L, 10 mg/L, 11 mg/L, 12 mg/L, 13 mg/L, or 14 mg/L. In some embodiments, the antioxidant enzyme cofactor (e.g., a selenium compound) is added in an amount of about 0.01 mg/L to about 0.02 mg/L, such as about any of 0.011 mg/L, 0.012 mg/L, 0.013 mg/L, 0.014 mg/L, 0.015 mg/L, 0.016 mg/L, 0.017 mg/L, 0.018 mg/L, or 0.019 mg/L. In some embodiments, the lipid precursor (e.g., ethanolamine) is added in an amount of about 1 mg/L to about 10 mg/L, such as about any of 2 mg/L, 3 mg/L, 4 mg/L, 5 mg/L, 6 mg/L, 7 mg/L, 8 mg/L, or 9 mg/L. In some embodiments, the carrier protein (e.g., albumin) is added in an amount of about 100 mg/L to about 1000 mg/L, such as about any of 200 mg/L, 300 mg/L, 400 mg/L, 500 mg/L, 600 mg/L, 700 mg/L, 800 mg/L, or 900 mg/L. In some embodiments, the serum (e.g., AB serum) is added to the culture medium in an amount of about 0.5% (v/v) to about 5% (v/v), such as about any of 0.5% (v/v), 1% (v/v), 1.5% (v/v), 2% (v/v), 2.5% (v/v), 3% (v/v), 3.5% (v/v), 4% (v/v), 4.5% (v/v), or 5% (v/v).

### B. Harvesting

In certain aspects, the methods of producing a population of cells comprising γδ T cells comprise harvesting the population of cells comprising γδ T cells following culturing. In some embodiments, cells are harvested at other stages of the methods provided herein, such as for transduction, and the harvesting techniques described herein are also applicable to perform harvesting techniques at such stages. In some embodiments, the harvesting step is not performed to obtain the population of cell comprising the γδ T cells. One of skill in the art will readily appreciate whether a harvesting step is necessary in the methods provided herein, such as based on a downstream use of the population of cells comprising the γδ T cells.

Harvesting techniques, including culturing techniques for expanding γδ T cells, are well known in the art. See, e.g., Segeritz et al., Basic Science Methods for Clinical Researchers, 2017; and Fundamental Techniques in Cell Culture, 3rd Edition, 2016, the contents of which are incorporated herein by reference in their entirety. In some embodiments, the harvesting comprises obtaining the population of cells comprising γδ T cells in a solution, e.g., suspending the population of cells comprising γδ T cells. In some embodiments, the harvesting comprises concentrating the population of cells comprising γδ T cells, such as by using centrifugation. In some embodiments, the harvesting comprises removing at least about any of about 50%, about 60%, about 70%, about 80%, about 85%, about 87%, about 90%, about 91%, about 92%, about 93%, about 94%, about 95%, about 96%, about 97%, about 98%, about 99%, or about 100% of the culture medium from the population of cells comprising γδ T cells, such as the culture medium using during the culturing. In some embodiments, the harvesting comprises resuspending the cells in a solution (such as in DMSO) for cryopreservation. In some embodiments, the harvesting comprises resuspending the cells in a solution (such as a pharmaceutically acceptable excipient) for administration to a subject (such as a human patient) in need thereof.

In some embodiments, the harvesting is performed, such as performed at a time, based on achieving one or more characteristics of a produced population of cells comprising γδ T cells, including one or more characteristics of the γδ T cells. In some embodiments, the one or more characteristics include the number of cells (e.g., number of produced γδ T cells), degree of expansion (e.g., degree of expansion of γδ T cells), or a phenotype, such as based on lineage, activation, exhaustion, memory, and/or antigen presentation. Additional details regarding characteristics, including phenotypes of population of cells comprising γδ T cells, are provided elsewhere in the present disclosure. In some embodiments, the harvesting is performed on about day 7 to about day 28, including on about day 7 to about day 18, or about day 10 to about day 14. In some embodiments, the harvesting is performed on about any of the following days: day 7, day 8, day 9, day 10, day 11, day 12, day 13, day 14, day 15, day 16, day 17, day 18, day 19, day 20, day 21, day 22, day 23, or day 24.

### C. Steps Performed Prior to Culturing

In certain embodiments, the methods provided herein comprise one or more additional step(s) performed prior to the culturing of an isolated pool of mixed immune cells depleted of αβ T cells (such as an isolated pool of PBMCs), or the derivative thereof, in the culture medium. In some embodiments, the method further comprises a step of αβ T cell depletion. In some embodiments, the method further comprises a step of obtaining, such as isolating, a pool of mixed immune cells or a pool of mixed immune cells depleted of αβ T cells.

### i. αβ T Cell Depletion

In certain embodiments, the method comprises performing a step of αβ T cell depletion on an isolated pool of mixed immune cells to obtain a population of cells depleted of αβ T cells. In some embodiments, αβ T cells are removed from a pool of mixed immune cells by negative selection or using a suitable isolation technique.

In some embodiments, the step of αβ T cell depletion comprises an antibody-based depletion. In some embodiments, the step of αβ T cell depletion comprises (a) subjecting the pool of mixed immune cells to an anti-TCR α/β antibody; and (b) separating the antibody-conjugated αβ TCR+ T cells from other cells in the pool of mixed immune cells to obtain an αβ T cell depleted population of cells. In some embodiments, the separating is performed by a cell sorting technique (including, but not limited to, magnetic-activated cell sorting (MACS) or fluorescence-activated cell sorting (FACS)).

In some embodiments, the population of cells obtained from αβ T cells depletion (an isolated pool of mixed immune cells depleted of αβ T cells) is cryopreserved for a period of time prior to performing a culturing step taught herein. Cryopreservation techniques are described in more detail elsewhere in the present disclosure.

### ii. Isolating/Obtaining a Pool of Mixed Immune Cells

In certain embodiments, the method comprises obtaining an isolated pool of mixed immune cells, including an isolated pool of mixed immune cells depleted of αβ T cells. In some embodiments, the pool of mixed immune cells depleted of αβ T cells is an isolated pool of PBMCs. As described herein, an isolated pool of PBMCs may comprise additional cells, e.g., comprises a PBMC purity of at least about 85%. In some embodiments, one or more precursors (such as prior to αβ T cell depletion) comprises the PBMCs, such as obtaining isolated PBMCs from an individual.

In some embodiments, the method comprises isolating a pool of mixed immune cells, such as PBMCs, from a donor (such as a human donor, including a healthy human donor or a human patient with a disease or disorder). In some embodiments, the isolated pool of mixed immune cells, such as PBMCs, from a donor (such as a human donor) are from a single collection from the donor, e.g., a single blood draw or cell collection. In some embodiments, the isolated pool of mixed immune cells, such as PBMCs, from a donor (such as a human donor) are from a plurality of collections from the donor. In some embodiments, the isolated pool of mixed immune cells, such as PBMCs, are pooled from one or more donors (such as human donors), e.g., a plurality of collections from a single donor, or one or more collections from a plurality of donors. In some embodiments, the pooled immune cells from one or more sources, such as one or more collections or one or more donors, is performed after of T cell depletion. In some embodiments, the sample collected from a donor (such as a human donor) comprises peripheral blood cells such as from apheresis, buffy coat, whole blood, LRSC, or leukopak. In some embodiments, the isolated pool of mixed immune cells, such as PBMCs, are isolated from tumor-infiltrating or tissue-resident immune cells, natural or induced pluripotent/stem/progenitor cells, ascites, or other human body fluids or bone marrow cells.

In some embodiments, the isolating comprising leukapheresis. In some embodiments, the isolating is performed on blood from the donor (such as a human donor). In some embodiments, the method further comprises obtaining blood from the donor (such as a human donor).

In some embodiments, the donor is a human individual. In some embodiments, the donor is a healthy individual (e.g., does not have the disease or condition for which the produced cell population comprising γδ T cells is used to treat). In some embodiments, the donor is an individual suspected or diagnosed as having a disease, disorder, and/or medical condition. In some embodiments, the donor is the intended recipient for the produced cell population comprising γδ T cells. In some embodiments, the donor is not the intended recipient for the produced cell population comprising γδ T cells. One skilled in the art will readily recognize that where the donor and recipient are different individuals, the therapy is characterized as allogeneic, and where the donor and recipient are the same individuals, the therapy is characterized as autologous.

### D. Transduction

In certain aspects, the methods provided herein comprise a step of transducing cells of the population with a transgene. In some embodiments, the transgene is a chimeric antigen receptor (CAR).

Various techniques are known for transducing cells to modify gene expression. See, e.g., Bilal et al., Immunol Cell Biol, 93, 2015, the contents of which are incorporated herein by reference in their entirety. Generally speaking, transduction involves introducing an agent such as an exogenous agent (for example, via a viral-mediated delivery, e.g., a virus or viral vector) that will modify gene expression of the cell. In some embodiments, transduction involves delivering a gene or a modified gene, including a transgene, via a retrovirus. Examples of virus or viral vectors used for viral-mediated transgene delivery include: adenoviruses, adeno-associated viruses (AAV), alphaviruses, flaviviruses, herpes simplex viruses (HSV), measles viruses, rhabdoviruses, retroviruses, lentiviruses, Newcastle disease virus (NDV), poxviruses, and picornaviruses. Other techniques for modifying cells are known and may be used in the methods taught herein. For example, non-viral approaches are known and include delivery of material via plasmids, nanoparticles, lipoplexes, liposomes, lipid-conjugation, or exosomes. In some embodiments, a gene, such as a transgene, is delivered to a cell. In some embodiments, the gene, such as a transgene, is further modified. In some embodiments, the modified gene, such as a modified transgene, is a CAR.

In some embodiments, the cell is modified via delivery of a nucleic acid editing system, such as CRISPR/Cas9, TALENS, ZFN, etc.

In some embodiments, the method of transduction and associated cell recovery and culturing comprises a static technique, such as static bag or plate transduction. In some embodiments, the method of transduction and associated cell recovery and culturing comprises a spinoculation technique, such as Sepax spinoculation, plate or bag spinoculation. See, e.g., Remley et al., J Translational Medicine, 19, 2021. In some embodiments, the bag or plate is coated. In some embodiments, the coating is RetroNectin.

In some embodiments, the method comprises two or more culturing steps, wherein a step of transduction occurs after a first culturing step and prior to a second culturing step. In such embodiments, the first culturing step of about 0 to about 4 days. In some embodiments, the step of transducing is performed on about day 4. In some embodiments, the second culturing step is about 0 to about 10 days.

The present cell modifications encompassed by the disclosure are numerous and may be guided by the use of the produced populations of cells. For example, in some embodiments, the transducing comprises introducing a CAR. In some embodiments, the CAR specifically recognizes globohexaosylceramide, AKR1C3, SSEA-4, or TROP-2.

### E. Cryopreservation

In some embodiments, the methods provided herein comprise one or more cryopreservation steps, such as for cryopreserving cells at different stages of the methods. In some embodiments, cryopreservation is performed on the isolated pool of mixed immune cells or the isolated pool of mixed immune cells depleted of αβ T cells, such as PBMCs, or the derivative thereof (e.g., a population of PBMCs depleted of αβ T cells). In some embodiments, cryopreservation is performed on the harvested population of cells comprising γδ T cells. In some embodiments, cryopreservation is performed on the harvested population of cells comprising γδ T cells, wherein the population of cells comprising γδ T cells was subjected to a step of transduction.

Cryopreservation techniques are well known in the art. In some embodiments, the cryopreservation technique comprises admixing cells, such as a population of PBMCs depleted of αβ T cell or a population of cells comprising γδ T cells produced using the methods described herein, with a freezing medium and placing the resulting composition in cryogenic storage, such as liquid nitrogen freezers (−195° C.) or ultra-low temperature freezers (−65° C. −80° C., or −120° C.) for storage of up to or at least about any of 1 day, 1 week, 2 weeks, 3 weeks, 1 month, 2 months, 3 months, 4 months, 5 months, 6 months, 1 year, 2 years, 3 years, or 5 years. The freezer medium can contain dimethyl sulfoxide (DMSO), and/or sodium chloride (NaCl), and/or dextrose, and/or dextran sulfate and/or hydroyethyl starch (HES) with physiological pH buffering agents to maintain pH between about 6.0 to about 6.5, about 6.5 to about 7.0, about 7.0 to about 7.5, about 7.5 to about 8.0 or about 6.5 to about 7.5. In some embodiment, the freezer medium comprises CryoStor CS10.

The cryopreserved cells may retain their functionality and can be thawed for later use dependent on the stage in the method at which cryopreservation occurred. For example, in some embodiments, the population of mixed immune cells depleted of αβ T cell may be cryopreserved and later thawed to continue culturing as taught herein to produce a population of cells comprising γδ T cells. In some embodiments, the produced population of cells comprising γδ T cells may be cryopreserved and later thawed for a method of use described herein, e.g., a method of treatment.

In some embodiments, the cryopreservation comprises flushing cell plates and/or bags (such as GREX plates and/or bags) with culture medium, and transferring the cells, such as a population of cells comprising γδ T cells, in the culture medium to a container, such as a 50 ml conical tube. In some embodiments, the resulting cell population is counted. In some embodiments, the container is subjected to centrifugation to pellet the cells, followed by removal of the supernatant. In some embodiments, the obtained cell pellet is resuspended in CryoStor CS10 freezing medium at 4° C. In some embodiments, the resuspended cells are placed in cell freezing containers and stored at −80° C. for a period of time (such as overnight) before being transferred to long-term storage (such as in liquid nitrogen).

### F. Exemplary Methods

In some embodiments, there is provided a method for producing a population of cells comprising γδ T cells, the method comprising: culturing an isolated pool of mixed immune cells depleted of αβ T cells, such as PBMCs depleted of αβ T cells, or a derivative thereof, in a culture medium comprising: (a) a basal medium; (b) IL-15; and (c) the following: (i) insulin or a derivative thereof; (ii) transferrin; (iii) sodium selenite; (iv) ethanolamine; and (v) albumin or a derivative thereof; and, optionally, harvesting the population of cells comprising γδ T cells. In some embodiments, the IL-15 is human IL-15, such as recombinant human IL-15. In some embodiments, the recombinant human IL-15 is added to the culture medium in an amount of about 10 ng/mL. In some embodiments, the insulin is a human insulin. In some embodiments, the human insulin is added to the culture medium in an amount of about 20 mg/L. In some embodiments, the transferrin is a human transferrin. In some embodiments, the human transferrin is added to the culture medium in an amount of about 11 mg/L. In some embodiments, the sodium selenite is added to the culture medium in an amount of about 0.0134 mg/L. In some embodiments, the ethanolamine is added to the culture medium in an amount of about 4 mg/L. In some embodiments, the albumin is a human albumin. In some embodiments, the human albumin is added to the culture medium in an amount of about 400 mg/L. In some embodiments, the culture medium comprises ITSEA. In some embodiments, the culture medium further comprises a serum. In some embodiments, the serum is a human AB serum. In some embodiments, the human serum is added to the culture medium in an amount of about 1% (v/v). In some embodiments, the basal culture medium is NK MACS medium. In some embodiments, the culturing is for about 14 days. In some embodiments, the produced population of cells comprises γδ T cells has a γδ T cell purity of at least about 85%, such as at least about 95%. In some embodiments, the produced population of cells comprises γδ T cells comprising vδ2 γδ T cells, and, optionally, vδ1 γδ T cells. In some embodiments, the population of cells comprising γδ T cells has a vδ2 γδ T cell purity of at least about 70% relative to total T cells, such as γδ T cells, in the population. In some embodiments, the produced population of cells comprising γδ T cells has a vδ2 γδ T cell purity of at least about 95% relative to total T cells, such as γδ T cells, in the population. In some embodiments, the produced population of cells comprising γδ T cells comprises less than about 5% of NK cells, such as less than about 2.5% of NK cells. In some embodiments, the produced population of cells comprising γδ T cells comprises γδ T cells comprising a CAR. In some embodiments, the CAR specifically recognizes globohexaosylceramide, AKR1C3, SSEA-4, or TROP-2. In some embodiments, the method further comprises cryopreserving the produced population of cells comprising γδ T cells following the harvesting step.

In some embodiments, there is provided a method for producing a population of cells comprising γδ T cells, the method comprising: culturing an isolated pool of mixed immune cells depleted of αβ T cells, such as PBMCs depleted of αβ T cells, or a derivative thereof, in a culture medium comprising: (a) a basal culture medium (b) IL-15; and (c) the following: (i) insulin or a derivative thereof; (ii) transferrin; (iii) sodium selenite; (iv) ethanolamine; and (v) albumin or a derivative thereof; performing a step of γδ T cell transduction; and, optionally, harvesting the population of cells comprising γδ T cells. In some embodiments, the IL-15 is human IL-15, such as recombinant human IL-15. In some embodiments, the recombinant human IL-15 is added to the culture medium in an amount of about 10 ng/ml. In some embodiments, the insulin is a human insulin. In some embodiments, the human insulin is added to the culture medium in an amount of about 20 mg/L. In some embodiments, the transferrin is a human transferrin. In some embodiments, the human transferrin is added to the culture medium in an amount of about 11 mg/L. In some embodiments, the sodium selenite is added to the culture medium in an amount of about 0.0134 mg/L. In some embodiments, the ethanolamine is added to the culture medium in an amount of about 4 mg/L. In some embodiments, the albumin is a human albumin. In some embodiments, the human albumin is added to the culture medium in an amount of about 400 mg/L. In some embodiments, the culture medium comprises ITSEA. In some embodiments, the culture medium further comprises a serum. In some embodiments, the serum is a human AB serum. In some embodiments, the human serum is added to the culture medium in an amount of about 1% (v/v). In some embodiments, the basal culture medium is NK MACS medium. In some embodiments, the culturing is for about 14 days. In some embodiments, the culturing step comprises a first culturing step of about 0 to about 4 days, with the transducing performed on about day 4. In some embodiments, the method comprises a second culturing step of about 0 to about 10 days following transduction. In some embodiments, the transducing comprises introducing a CAR to the cells. In some embodiments, the produced population of cells comprises γδ T cells has a γδ T cell purity of at least about 85%, such as at least about 95%. In some embodiments, the produced population of cells comprises γδ T cells comprising vδ2 γδ T cells, and, optionally, vδ1 γδ T cells. In some embodiments, the population of cells comprising γδ T cells has a vδ2 γδ T cell purity of at least about 70% relative to total T cells, such as γδ T cells, in the population. In some embodiments, the produced population of cells comprising γδ T cells has a vδ2 γδ T cell purity of at least about 95% relative to total T cells, such as γδ T cells, in the population. In some embodiments, the produced population of cells comprising γδ T cells comprises less than about 5% of NK cells, such as less than about 2.5% of NK cells. In some embodiments, the produced population of cells comprising γδ T cells comprises γδ T cells comprising a CAR. In some embodiments, the CAR specifically recognizes globohexaosylceramide, AKR1C3, SSEA-4, or TROP-2. In some embodiments, the method further comprises cryopreserving the produced population of cells comprising γδ T cells following the harvesting step.

In some embodiments, there is provided a method for producing a population of cells comprising γδ T cells, the method comprising: performing a step of αβ T cell depletion on an isolated pool of mixed immune cells, such as PBMCs; culturing the isolated pool of mixed immune cells depleted of αβ T cells, or a derivative thereof, in a culture medium comprising: (a) a basal culture medium; (b) IL-15; and (c) the following: (i) insulin or a derivative thereof; (ii) transferrin; (iii) sodium selenite; (iv) ethanolamine; and (v) albumin or a derivative thereof; and, optionally, harvesting the population of cells comprising γδ T cells. In some embodiments, the IL-15 is human IL-15, such as recombinant human IL-15. In some embodiments, the recombinant human IL-15 is added to the culture medium in an amount of about 10 ng/ml. In some embodiments, the insulin is a human insulin. In some embodiments, the human insulin is added to the culture medium in an amount of about 20 mg/L. In some embodiments, the transferrin is a human transferrin. In some embodiments, the human transferrin is added to the culture medium in an amount of about 11 mg/L. In some embodiments, the sodium selenite is added to the culture medium in an amount of about 0.0134 mg/L. In some embodiments, the ethanolamine is added to the culture medium in an amount of about 4 mg/L. In some embodiments, the albumin is a human albumin. In some embodiments, the human albumin is added to the culture medium in an amount of about 400 mg/L. In some embodiments, the culture medium comprises ITSEA. In some embodiments, the culture medium further comprises a serum. In some embodiments, the serum is a human AB serum. In some embodiments, the human serum is added to the culture medium in an amount of about 1% (v/v). In some embodiments, the basal culture medium is NK MACS medium. In some embodiments, the culturing is for about 14 days. In some embodiments, the step of αβ T cell depletion comprises an antibody-based depletion. In some embodiments, the step of αβ T cell depletion comprises (a) subjecting the pool of mixed immune cells to an anti-TCR α/β antibody; and (b) separating the antibody-conjugated αβ TCR+ T cells from other cells in the pool of mixed immune cells to obtain an αβ T cell depleted population of cells. In some embodiments, the produced population of cells comprises γδ T cells has a γδ T cell purity of at least about 85%, such as at least about 95%. In some embodiments, the produced population of cells comprises γδ T cells comprising vδ2 γδ T cells, and, optionally, vδ1 γδ T cells. In some embodiments, the population of cells comprising γδ T cells has a vδ2 γδ T cell purity of at least about 70% relative to total T cells, such as γδ T cells, in the population. In some embodiments, the produced population of cells comprising γδ T cells has a vδ2 γδ T cell purity of at least about 95% relative to total T cells, such as γδ T cells, in the population. In some embodiments, the produced population of cells comprising γδ T cells comprises less than about 5% of NK cells, such as less than about 2.5% of NK cells. In some embodiments, the produced population of cells comprising γδ T cells comprises γδ T cells comprising a CAR. In some embodiments, the CAR specifically recognizes globohexaosylceramide, AKR1C3, SSEA-4, or TROP-2. In some embodiments, the method further comprises cryopreserving the produced population of cells comprising γδ T cells following the harvesting step.

In some embodiments, there is provided a method for producing a population of cells comprising γδ T cells, the method comprising: performing a step of αβ T cell depletion on an isolated pool of mixed immune cells, such as PBMCs; culturing the isolated pool of mixed immune cells depleted of αβ T cells, or a derivative thereof, in a culture medium comprising: (a) a basal culture medium; (b) IL-15; and (c) the following: (i) insulin or a derivative thereof; (ii) transferrin; (iii) sodium selenite; (iv) ethanolamine; and (v) albumin or a derivative thereof; performing a step of γδ T cell transduction; and, optionally, harvesting the population of cells comprising γδ T cells. In some embodiments, the IL-15 is human IL-15, such as recombinant human IL-15. In some embodiments, the recombinant human IL-15 is added to the culture medium in an amount of about 10 ng/mL. In some embodiments, the insulin is a human insulin. In some embodiments, the human insulin is added to the culture medium in an amount of about 20 mg/L. In some embodiments, the transferrin is a human transferrin. In some embodiments, the human transferrin is added to the culture medium in an amount of about 11 mg/L. In some embodiments, the sodium selenite is added to the culture medium in an amount of about 0.0134 mg/L. In some embodiments, the ethanolamine is added to the culture medium in an amount of about 4 mg/L. In some embodiments, the albumin is a human albumin. In some embodiments, the human albumin is added to the culture medium in an amount of about 400 mg/L. In some embodiments, the culture medium comprises ITSEA. In some embodiments, the culture medium further comprises a serum. In some embodiments, the serum is a human AB serum. In some embodiments, the human serum is added to the culture medium in an amount of about 1% (v/v). In some embodiments, the culture medium comprises a basal culture medium. In some embodiments, the basal culture medium is NK MACS medium. In some embodiments, the culturing is for about 14 days. In some embodiments, the step of αβ T cell depletion comprises an antibody-based depletion. In some embodiments, the step of αβ T cell depletion comprises (a) subjecting the pool of mixed immune cells to an anti-TCR α/β antibody; and (b) separating the antibody-conjugated αβ TCR+ T cells from other cells in the pool of mixed immune cells to obtain an αβ T cell depleted population of cells. In some embodiments, the culturing step comprises a first culturing step of about 0 to about 4 days, with the transducing performed on about day 4. In some embodiments, the method comprises a second culturing step of about 0 to about 10 days following transduction. In some embodiments, the transducing comprises introducing a CAR to the cells. In some embodiments, the produced population of cells comprises γδ T cells has a γδ T cell purity of at least about 85%, such as at least about 95%. In some embodiments, the produced population of cells comprises γδ T cells comprising vδ2 γδ T cells, and, optionally, vδ1 γδ T cells. In some embodiments, the population of cells comprising γδ T cells has a vδ2 γδ T cell purity of at least about 70% relative to total T cells, such as γδ T cells, in the population. In some embodiments, the produced population of cells comprising γδ T cells has a vδ2 γδ T cell purity of at least about 95% relative to total T cells, such as γδ T cells, in the population. In some embodiments, the produced population of cells comprising γδ T cells comprises less than about 5% of NK cells, such as less than about 2.5% of NK cells. In some embodiments, the produced population of cells comprising γδ T cells comprises γδ T cells comprising a CAR. In some embodiments, the CAR specifically recognizes globohexaosylceramide, AKR1C3, SSEA-4, or TROP-2. In some embodiments, the method further comprises cryopreserving the produced population of cells comprising γδ T cells following the harvesting step.

### III. Populations of Cells Comprising γδ T Cells

Provided herein, in certain aspects are populations of cells comprising γδ T cells produced using the methods described herein. In some embodiments, the populations of cells comprising γδ T cells display functional properties known to be important for supporting effective adoptive cellular therapy activity. For example, T cells of the memory or naïve T cell lineages display improved engraftment, expansion, and anti-tumor activity than terminal effector T cells (see, e.g., Gattinoni et al. (2005) J Clin Invest 115(6): 1616-1626; Janelle and Delisle (2021), Cancers (Basel) 13(4): 598; and López-Cantillo et al (2022), Front Immunol 13: 878209). Similarly, the functional properties of T cell exhaustion and senescence are important indicators of downstream therapeutic effectiveness, such that cells displaying high levels of senescence markers (e.g., high levels of KLRG1 and CD57 and low levels of CD27 or CD28) or exhaustion markers (e.g., high levels of PD-1, CTLA-4, LAG-3, TIM-3, TIGIT, etc. and low levels of perforin, TNF-α, IL-2, IFN-γ, Granzyme B, etc.) display significantly lower T cell expansion or anti-tumor activity upon adoptive cellular transfer. In some embodiments, the γδ T cells are not of the terminal effector T cell lineage. In some embodiments, the γδ T cells are not senescent. In some embodiments, the γδ T cells are not functionally exhausted.

In some embodiments, the population of cells comprises about 1×10³ cells to about 1×10¹⁰ cells. In some embodiments, the population of cells comprises at least about any of 1×10³ cells, 2×10³ cells, 3×10³ cells, 4×10³ cells. 5×10³ cells, 6×10³ cells, 7×10³ cells, 8×10³ cells, 9×10³ cells, 1×10⁴ cells, 2×10⁴ cells, 3×10⁴ cells, 4×10⁴ cells, 5×10⁴ cells, 6×10⁴ cells, 7×10⁴ cells, 8×10⁴ cells, 9×10⁴ cells, 1×10⁵ cells, 2×10⁵ cells, 3×10⁵ cells, 4×10⁵ cells, 5×10⁵ cells, 6×10⁵ cells, 7×10⁵ cells, 8×10⁵ cells, 9×10⁵ cells, 1×10⁶ cells, 2×10⁶ cells, 3×10⁶ cells, 4×10⁶ cells, 5×10⁶ cells, 6×10⁶ cells, 7×10⁶ cells, 8×10⁶ cells, 9×10⁶ cells, 1×10⁷ cells, 2×10⁷ cells, 3×10⁷ cells, 4×10⁷ cells, 5×10⁷ cells, 6×10⁷ cells, 7×10⁷ cells, 8×10⁷ cells, 9×10⁷ cells, 1×10⁸ cells, 2×10⁸ cells, 3×10⁸ cells, 4×10⁸ cells, 5×10⁸ cells, 6×10⁸ cells, 7×10⁸ cells, 8×10⁸ cells, 9×10⁸ cells, 1×10⁹ cells, 2×10⁹ cells, 3×10⁹ cells, 4×10⁹ cells, 5×10⁹ cells, 6×10⁹ cells, 7×10⁹ cells, 8×10⁹ cells, 9×10⁹ cells, or 1×10¹⁰ cells. In some embodiments, the population of cells comprises about 1×10³ γδ T cells to about 1×10¹⁰ γδ T cells. In some embodiments, the population of cells comprises at least about any of 1×10³ γδ T cells, 2×10³ γδ T cells, 3×10³ γδ T cells, 4×10³ γδ T cells, 5×10³ γδ T cells. 6×10³ γδ T cells, 7×10³ γδ T cells, 8×10³ γδ T cells, 9×10³ γδ T cells, 1×10⁴ γδ T cells, 2×10⁴ γδ T cells, 3×10⁴ γδ T cells, 4×10⁴ γδ T cells, 5×10⁴ γδ T cells, 6×10⁴ γδ T cells, 7×10⁴ γδ T cells, 8×10⁴ γδ T cells, 9×10⁴ γδ T cells, 1×10⁵ γδ T cells, 2×10⁵ γδ T cells, 3×10⁵ γδ T cells, 4×10⁵ γδ T cells, 5×10⁵ γδ T cells, 6×10⁵ γδ T cells, 7×10⁵ γδ T cells, 8×10⁹ γδ T cells, 9×10⁵ γδ T cells, 1×10⁶ γδ T cells, 2×10⁶ γδ T cells, 3×10⁶ γδ T cells, 4×10⁶ γδ T cells, 5×10⁶ γδ T cells, 6×10⁶ γδ T cells, 7×10⁶ γδ T cells, 8×10⁶ γδ T cells, 9×10⁶ γδ T cells, 1×10⁷ γδ T cells, 2×10⁷ γδ T cells, 3×10⁷ γδ T cells, 4×10⁷ γδ T cells, 5×10⁷ γδ T cells, 6×10⁷ γδ T cells, 7×10⁷ γδ T cells, 8×10⁷ γδ T cells, 9×10⁷ γδ T cells, 1×10⁸ γδ T cells, 2×10⁸ γδ T cells, 3×10⁸ γδ T cells, 4×10⁸ γδ T cells, 5×10⁸ γδ T cells, 6×10⁸ γδ T cells, 7×10⁸ γδ T cells, 8×10⁸ γδ T cells, 9×10⁸ γδ T cells, 1×10⁹ γδ T cells, 2×10⁹ γδ T cells, 3×10⁹ γδ T cells, 4×10⁹ γδ T cells, 5×10⁹ γδ T cells, 6×10⁹ γδ T cells, 7×10⁹ γδ T cells, 8×10⁹ γδ T cells, 9×10⁹ γδ T cells, or 1×10¹⁰ γδ T cells.

In some embodiments, the population of cells comprising γδ T cells produced using the methods provided herein has a γδ T cell purity of at least about 85%, such as at least about any of 86%, 87%, 88%, 89%, 90%, 91%, 92%, 93%, 94%, 95%, 95.5%, 96%, 96.5%, 97%, 97.5%, 98%, 98.5%, 99%, 99.5%, or 100%. In some embodiments, the population of cells comprising γδ T cells produced using the methods provided herein comprises vδ2 γδ T cells, and, optionally, vδ1 γδ T cells. In some embodiments, the population of cells comprising γδ T cells has a vδ2 γδ T cell purity, relative to total T cells, such as γδ T cells, in the population, of at least about 70%, such as at least about any of 75%, 80%, 85%, 90%, or 95%. In some embodiments, the population of cells comprising γδ T cells produced using the methods provided herein has a vδ2 γδ T cell purity, relative to total T cells, such as γδ T cells, in the population, of at least about 95%, such as at least about any of 95.5%, 96%, 96.5%, 97%, 97.5%, 98%, 98.5%, 99%, 99.5%, or 100%. In any embodiments described herein, the vδ2 γδ T cell purity is relative to total T cells in the population. In any embodiments described herein, the vδ2 γδ T cell purity is relative to total γδ T cells in the population. In some embodiments, the population of cells comprising γδ T cells produced using the methods provided herein comprises less than about 15% of NK cells, such as less than about any of 14% of NK cells, 13% of NK cells, 12% of NK cells, 11% of NK cells, 10% of NK cells, 9% of NK cells, 8% of NK cells, 7% of NK cells, 6% of NK cells, 5% of NK cells, 4.5% of NK cells, 4% of NK cells, 3.5% of NK cells, 3% of NK cells, 2.5% of NK cells, 2% of NK cells, 1.5% of NK cells, 1% of NK cells, or 0.5% of NK cells.

In some embodiments, the population of cells comprising γδ T cells comprises less than about 10% vδ1 γδ T cells, such as less than about any of 9% vδ1 γδ T cells, 8% vδ1 γδ T cells, 7% vδ1 γδ T cells, 6% vδ1 γδ T cells, 5% vδ1 γδ T cells, 4% vδ1 γδ T cells, 3% vδ1 γδ T cells, 2% vδ1 γδ T cells, or 1% vδ1 γδ T cells.

In some embodiments, the population of cells comprising γδ T cells comprises less than about 1% αβ T cells, such as less than about any of 0.9% αβ T cells, 0.8% αβ cells, 0.7% αβ T cells, 0.6% αβ T cells, 0.5% αβ T cells, 0.4% αβ T cells, 0.3% αβ T cells, 0.2% αβ T cells, or 0.1% αβ T cells.

### IV. Pharmaceutical Compositions and Kits

In certain aspects, provided herein are pharmaceutical compositions comprising the populations of cells comprising γδ T cells produced using the methods described herein, and optionally a pharmaceutically acceptable carrier. Further provided by the present application are kits and composition useful for the methods provided herein, and kits and compositions obtained from the methods provided herein, e.g., kits and compositions of the populations of cells comprising γδ T cells produced using the methods described herein.

### A. Pharmaceutical Composition Formulation

In some embodiments, the pharmaceutical composition may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21st Edition, A. R. Gennaro; Lippincott, Williams & Wilkins, Baltimore, Md., 2006).

Examples of diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof.

Preservatives include, but are not limited to, antioxidants, chelating agents, antimicrobial preservatives, antifungal preservatives, alcohol preservatives, acidic preservatives, and/or other preservatives. Antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Antifungal preservatives include, but are not limited to, butyl paraben, methyl paraben, ethyl paraben, propyl paraben, benzoic acid, hydroxy benzoic acid, potassium benzoate, potassium sorbate, sodium benzoate, sodium propionate, and/or sorbic acid. Examples of alcohol preservatives include, but are not limited to, ethanol, polyethylene glycol, phenol, benzyl alcohol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN® II, NEOLONE™ KATHON™, and/or EUXYL®.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g. HEPES), magnesium hydroxide, aluminum hydroxide, alginic acid, pyrogen-free water, isotonic saline, Ringer's solution, ethyl alcohol, and/or combinations thereof. Lubricating agents may be selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

In some embodiments, the pharmaceutical composition is formulated to have a pH in the range of about 4.5 to about 9.0, including for example pH ranges of about any one of 5.0 to about 8.0, about 6.5 to about 7.5, or about 6.5 to about 7.0. In some embodiments, the pharmaceutical composition is formulated to have a pH in the range of about 4.5 to about 6.5. In some embodiments, the pharmaceutical composition is formulated to have a pH in the range of about 7.3 to about 7.5. In some embodiments, the pharmaceutical composition can be made to be isotonic with blood by the addition of a suitable tonicity modifier.

The pharmaceutical compositions described herein may be prepared by any method known or hereafter developed in the art of pharmacology. In general, such preparatory methods include the step of associating the populations of cells comprising γδ T cells produced using the methods described herein with an excipient and/or one or more other accessory ingredients. In some embodiments, the pharmaceutical composition may be prepared, packaged, and/or sold in bulk, as a single unit dose, and/or as a plurality of single unit doses. As used herein, a “unit dose” refers to a discrete amount of the pharmaceutical composition comprising a predetermined amount of the populations of cells comprising γδ T cells. The amount of the γδ T cells may generally be equal to the dosage of the γδ T cells which would be administered to an individual and/or a convenient fraction of such a dosage including, but not limited to, one-half or one-third of such a dosage.

### B. Administration

The pharmaceutical compositions to be used for in vivo administration are generally formulated as sterile, substantially isotonic, and in full compliance with all Good Manufacturing Practice (GMP) regulations of the U.S. Food and Drug Administration. In some embodiments, the composition is free of pathogens. For parenteral administration, the pharmaceutical composition can be in the form of liquid solutions, for example in physiologically compatible buffers such as Hank's solution or Ringer's solution.

In some embodiments, the pharmaceutical composition is formulated in accordance with routine procedures as a pharmaceutical composition adapted for parenteral administration, such as for intravenous, subcutaneous, peritoneal, intravitreal, or intratumoral administration. In some embodiments, the pharmaceutical composition is formulated in accordance with routine procedures as a pharmaceutical composition adapted for intravenous administration.

In some embodiments, the pharmaceutical composition is formulated in accordance with routine procedures as a pharmaceutical composition adapted for injection intravenously, intraperitoneally, subcutaneously, intramuscularly, intratumorally, intradermally, or intravitreally. Typically, compositions for injection are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form.

In some embodiments, the pharmaceutical composition is suitable for administration to a human. In some embodiments, the pharmaceutical composition is suitable for administration to a rodent (e.g., mice, rats) or non-human primates (e.g., Cynomolgus monkey). In some embodiments, the pharmaceutical composition is contained in a single-use vial, such as a single-use sealed vial. In some embodiments, the pharmaceutical composition is contained in a multi-use vial. In some embodiments, the pharmaceutical composition is contained in bulk in a container. In some embodiments, the pharmaceutical composition is cryopreserved.

Also provided are unit dosage forms of any of the populations of cells comprising γδ T cells produced using the methods described herein, or compositions (such as pharmaceutical compositions) thereof. These unit dosage forms can be stored in a suitable packaging in single or multiple unit dosages and may also be sterile and sealed. In some embodiments, the pharmaceutical composition is administered as a single dose. In some embodiments, the pharmaceutical composition is administered as multiple doses. In some embodiments, the pharmaceutical composition is administered once every 4 weeks, such as about any of 4 weeks, 5 weeks, 6 weeks, 7 weeks, 8 weeks, 9 weeks, 10 weeks, 11 weeks, 12 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 1 year, 2 years, 3 years, or more. In some embodiments, the pharmaceutical composition is administered no more than once a year.

In some embodiments, the pharmaceutical composition is administered in combination, or in an overall treatment regimen, with one or more therapies. In some embodiments, the pharmaceutical composition is administered simultaneously with one or more therapies. In some embodiments, the pharmaceutical composition is administered sequentially with one or more therapies. In some embodiments, the pharmaceutical composition is administered as a standalone therapy. In some embodiments, the pharmaceutical composition is administered after one or more therapies for a disease or disorder have failed to treat or cure the individual.

In some embodiments, the pharmaceutical composition is administered to treat an individual diagnosed with or suspected of having a cancer. In some embodiments, the pharmaceutical composition is administered in combination, or in an overall treatment regiment, with one or more anti-cancer therapies. In some embodiments, the pharmaceutical composition and the one or more anti-cancer therapies are administered simultaneously. In some embodiments, the pharmaceutical composition and the one or more anti-cancer therapies are administered sequentially. In some embodiments, the one or more anti-cancer therapies are selected from a chemotherapeutic agent, a cytokine, an immunotherapy, a radiotherapy, an anti-cancer vaccine, and a therapeutic antibody. For example, in some embodiments, the one or more anti-cancer therapies may comprise anti-PD-1, anti-PDL-1, and/or anti-PDL-2 monoclonal antibodies or biologically active fragments thereof. In some embodiments, the pharmaceutical composition is administered as an adjuvant therapy after the individual diagnosed with a cancer has undergone surgical treatment. In some embodiments, the cancer is a solid tumor malignancy. In some embodiments, the cancer is a liquid cancer, such as a hematological cancer. In some embodiments, the cancer can range from early-to late-stage cancer.

In some embodiments, the pharmaceutical composition is administered to treat an individual diagnosed with or suspected of having an autoimmune disorder. In some embodiments, the pharmaceutical composition is administered in combination, or in an overall treatment regiment, with one or more autoimmune treatments. In some embodiments, the pharmaceutical composition and one or more autoimmune treatments are administered simultaneously. In some embodiments, the pharmaceutical composition and one or more autoimmune treatments are administered sequentially. In some embodiments, the one or more autoimmune treatments are selected from corticosteroid agents, immunosuppressant agents including high-dose, anti-inflammatory agents, gene therapy agents. In some embodiments, the pharmaceutical composition and the corticosteroid or immunosuppressant agents are not administered simultaneously. In some embodiments, the pharmaceutical composition is administered as an adjuvant therapy after the individual diagnosed with an autoimmune disorder has undergone surgical treatment. In some embodiments, the individual may further use pain-management agents or physical therapy.

In some embodiments, the pharmaceutical composition is administered as a prophylaxis or to treat an individual having or at risk of developing an infectious disease (such as a bacterial, fungal, viral, or parasitic disease). In some embodiments, the pharmaceutical composition is administered in combination, or in an overall treatment regiment, with one or more infectious disease treatments, including prophylaxis treatments. In some embodiments, the pharmaceutical composition and the one or more infectious disease treatments are administered simultaneously. In some embodiments, the pharmaceutical composition and the one or more infectious disease treatments are administered sequentially. In some embodiments, the one or more infectious disease treatments are administered for up to 100 days before and/or after administration of the pharmaceutical composition described herein. In some embodiments, the infectious disease treatments, including prophylaxis treatments, are selected from antibiotic agents, antiviral agents, antifungal agents, or vaccines.

In some embodiments, the pharmaceutical composition prevents, reduces, ameliorates, treats, or cures any one of the diseases or disorders described herein. In some embodiments, the pharmaceutical composition does not induce GvHD. In some embodiments, the pharmaceutical composition does not induce cytokine release syndrome. In some embodiments, the pharmaceutical composition does not induce neurotoxicity.

### C. Unit Dosage

In some embodiments, pharmaceutical compositions described herein are administered at a dose of about 1.0×10⁵ to 1.0×10¹⁰ cells/kg. For example, in some embodiments, pharmaceutical compositions described herein are administered at a dose of about 4.0×10⁵ to 5.0×10⁵ cells/kg, 4.5×10⁵ to 5.5×10⁵ cells/kg, 5.0×10⁵ to 6.0×10⁵ cells/kg, 5.5×10⁵ to 6.5×10⁵ cells/kg, 6.0×10⁵ to 7.0×10⁵ cells/kg, 6.5×10⁵ to 7.5×10⁵ cells/kg, 7.0×10⁵ to 8.0×10⁵ cells/kg, 7.5×10⁵ to 8.5×10⁵ cells/kg, 8.0×10⁵ to 9.0×10⁵ cells/kg, 8.5×10⁵ to 9.5×10⁵ cells/kg, 9.0×10⁵ to 1.0×10⁶ cells/kg. In certain embodiments, the pharmaceutical composition is administered at a dose of about 7.0×10⁷ cells/kg, about 8.0×10⁷ cells/kg, about 9.0×10⁷ cells/kg, about 1.0×10⁸ cells/kg, about 2.0×10⁸ cells/kg, about 3.0×10⁸ cells/kg, about 4.0×10 cells/kg, about 5.0×10⁸ cells/kg, about 6.0×10⁸ cells/kg, about 7.0×10⁸ cells/kg, about 8.0×10⁸ cells/kg, about 9.0×10⁸ cells/kg, about 1.0×10⁹ cells/kg, about 2.0×10⁹ cells/kg, about 3.0×10⁹ cells/kg, about 4.0×10⁹ cells/kg, about 5.0×10⁹ cells/kg, about 6.0×10⁹ cells/kg, about 7.0×10⁹ cells/kg, about 8.0×10⁹ cells/kg, about 9.0×10⁹ cells/kg, or about 1.0×10¹⁰ cells/kg. In one embodiment, the pharmaceutical composition is administered at a dose of about 7.5×10⁷ cells/kg. In certain embodiments, the pharmaceutical composition is administered at a dose of about 3.0 to 4.0×10⁸ cells. In certain embodiments, the pharmaceutical composition is administered at a dose of about 3.5 to 4.5×10⁸ cells. In certain embodiments, the pharmaceutical composition is administered at a dose of about 4.0 to 5.0×10⁸ cells. In certain embodiments, the pharmaceutical composition is administered at a dose of about 4.5 to 5.5×10⁸ cells. In certain embodiments, the pharmaceutical composition is administered at a dose of about 5.0 to 6.0×10⁸ cells. In certain embodiments, the pharmaceutical composition is administered at a dose of about 5.5 to 6.5×10⁸ cells. In certain embodiments, the pharmaceutical composition is administered at a dose of about 6.0 to 7.0×10⁸ cells. In certain embodiments, the pharmaceutical composition is administered at a dose of about 6.5 to 7.5×10⁸ cells. In certain embodiments, the pharmaceutical composition is administered at a dose of about 7.0 to 8.0×10⁸ cells. In certain embodiments, the pharmaceutical composition is administered at a dose of about 7.5 to 8.5×10⁸ cells. In certain embodiments, the pharmaceutical composition is administered at a dose of about 8.0 to 9.0×10⁸ cells. In certain embodiments, the pharmaceutical composition is administered at a dose of about 8.5 to 9.5×10⁸ cells. In certain embodiments, the pharmaceutical composition is administered at a dose of about 9.0 to 10.0×10⁸ cells. In certain embodiments, the pharmaceutical composition is administered at a dose of about 1.0 to 2.0×10⁹ cells. In certain embodiments, the pharmaceutical composition is administered at a dose of about 1.5 to 2.5×10⁹ cells. In certain embodiments, the pharmaceutical composition is administered at a dose of about 2.0 to 3.0×10⁹ cells. In certain embodiments, the pharmaceutical composition is administered at a dose of about 2.5 to 3.5×10⁹ cells. In certain embodiments, the pharmaceutical composition is administered at a dose of about 3.0 to 4.0×10⁹ cells. In certain embodiments, the pharmaceutical composition is administered at a dose of about 3.5 to 4.5×10⁹ cells. In certain embodiments, the pharmaceutical composition is administered at a dose of about 4.0 to 5.0×10⁹ cells. In certain embodiments, the pharmaceutical composition is administered at a dose of about 4.5 to 5.5×10⁹ cells. In certain embodiments, the pharmaceutical composition is administered at a dose of about 5.0 to 6.0×10⁹ cells. In certain embodiments, the pharmaceutical composition is administered at a dose of about 5.5 to 6.5×10⁹ cells. In certain embodiments, the pharmaceutical composition is administered at a dose of about 6.0 to 7.0×10⁹ cells. In certain embodiments, the pharmaceutical composition is administered at a dose of about 6.5 to 7.5×10⁹ cells. In certain embodiments, the pharmaceutical composition is administered at a dose of about 7.0 to 8.0×10⁹ cells. In certain embodiments, the pharmaceutical composition is administered at a dose of about 7.5 to 8.5×10⁹ cells. In certain embodiments, the pharmaceutical composition is administered at a dose of about 8.0 to 9.0×10⁹ cells. In certain embodiments, the pharmaceutical composition is administered at a dose of about 8.5 to 9.5×10⁹ cells. In certain embodiments, the pharmaceutical composition is administered at a dose of about 9.0 to 10.0×10⁹ cells.

In some embodiments, pharmaceutical compositions described herein are administered at a dose resulting in about 1.0×10⁵ to 1.0×10¹⁰ cells/kg in the subject (such as the human patient). For example, in some embodiments, pharmaceutical compositions described herein are administered at a dose resulting in about 4.0×10⁵ to 5.0×10⁵ cells/kg, 4.5×10⁵ to 5.5×10⁵ cells/kg, 5.0×10⁵ to 6.0×10⁵ cells/kg, 5.5×10⁵ to 6.5×10⁵ cells/kg, 6.0×10⁵ to 7.0×10⁵ cells/kg, 6.5×10⁵ to 7.5×10⁵ cells/kg, 7.0×10⁵ to 8.0×10⁵ cells/kg, 7.5×10⁵ to 8.5×10⁵ cells/kg, 8.0×10⁵ to 9.0×10⁵ cells/kg, 8.5×10⁵ to 9.5×10⁵ cells/kg, 9.0×10⁵ to 1.0×10⁶ cells/kg in the subject (such as the human patient). In certain embodiments, the pharmaceutical composition is administered at a dose resulting in about 7.0×10⁷ cells/kg, about 8.0×10⁷ cells/kg, about 9.0×10⁷ cells/kg, about 1.0×10⁸ cells/kg, about 2.0×10⁸ cells/kg, about 3.0×10⁸ cells/kg, about 4.0×10 cells/kg, about 5.0×10⁸ cells/kg, about 6.0×10⁸ cells/kg, about 7.0×10⁸ cells/kg, about 8.0×10⁸ cells/kg, about 9.0×10⁸ cells/kg, about 1.0×10⁹ cells/kg, about 2.0×10⁹ cells/kg, about 3.0×10⁹ cells/kg, about 4.0×10⁹ cells/kg, about 5.0×10⁹ cells/kg, about 6.0×10⁹ cells/kg, about 7.0×10⁹ cells/kg, about 8.0×10⁹ cells/kg, about 9.0×10⁹ cells/kg, or about 1.0×10¹⁰ cells/kg in the subject (such as the human patient). In one embodiment, the pharmaceutical composition is administered at a dose resulting in about 7.5×10⁷ cells/kg in the subject (such as the human patient). In certain embodiments, the pharmaceutical composition is administered at a dose resulting in about 3.0 to 4.0×10⁸ cells in the subject (such as the human patient). In certain embodiments, the pharmaceutical composition is administered at a dose resulting in about 3.5 to 4.5×10⁸ cells in the subject (such as the human patient). In certain embodiments, the pharmaceutical composition is administered at a dose resulting in about 4.0 to 5.0×10⁸ cells in the subject (such as the human patient). In certain embodiments, the pharmaceutical composition is administered at a dose resulting in about 4.5 to 5.5×10⁸ cells in the subject (such as the human patient). In certain embodiments, the pharmaceutical composition is administered at a dose resulting in about 5.0 to 6.0×10⁸ cells in the subject (such as the human patient). In certain embodiments, the pharmaceutical composition is administered at a dose resulting in about 5.5 to 6.5×10⁸ cells in the subject (such as the human patient). In certain embodiments, the pharmaceutical composition is administered at a dose resulting in about 6.0 to 7.0×10⁸ cells in the subject (such as the human patient). In certain embodiments, the pharmaceutical composition is administered at a dose resulting in about 6.5 to 7.5×10⁸ cells in the subject (such as the human patient). In certain embodiments, the pharmaceutical composition is administered at a dose resulting in about 7.0 to 8.0×10⁸ cells in the subject (such as the human patient). In certain embodiments, the pharmaceutical composition is administered at a dose resulting in about 7.5 to 8.5×10⁸ cells in the subject (such as the human patient). In certain embodiments, the pharmaceutical composition is administered at a dose resulting in about 8.0 to 9.0×10⁸ cells in the subject (such as the human patient). In certain embodiments, the pharmaceutical composition is administered at a dose resulting in about 8.5 to 9.5×10⁸ cells in the subject (such as the human patient). In certain embodiments, the pharmaceutical composition is administered at a dose resulting in about 9.0 to 10.0×10⁸ cells in the subject (such as the human patient). In certain embodiments, the pharmaceutical composition is administered at a dose resulting in about 1.0 to 2.0×10⁹ cells in the subject (such as the human patient). In certain embodiments, the pharmaceutical composition is administered at a dose resulting in about 1.5 to 2.5×10⁹ cells in the subject (such as the human patient). In certain embodiments, the pharmaceutical composition is administered at a dose resulting in about 2.0 to 3.0×10⁹ cells in the subject (such as the human patient). In certain embodiments, the pharmaceutical composition is administered at a dose resulting in about 2.5 to 3.5×10⁹ cells in the subject (such as the human patient). In certain embodiments, the pharmaceutical composition is administered at a dose resulting in about 3.0 to 4.0×10⁹ cells in the subject (such as the human patient). In certain embodiments, the pharmaceutical composition is administered at a dose resulting in about 3.5 to 4.5×10⁹ cells in the subject (such as the human patient). In certain embodiments, the pharmaceutical composition is administered at a dose resulting in about 4.0 to 5.0×10⁹ cells in the subject (such as the human patient). In certain embodiments, the pharmaceutical composition is administered at a dose resulting in about 4.5 to 5.5×10⁹ cells in the subject (such as the human patient). In certain embodiments, the pharmaceutical composition is administered at a dose resulting in about 5.0 to 6.0×10⁹ cells in the subject (such as the human patient). In certain embodiments, the pharmaceutical composition is administered at a dose resulting in about 5.5 to 6.5×10⁹ cells in the subject (such as the human patient). In certain embodiments, the pharmaceutical composition is administered at a dose resulting in about 6.0 to 7.0×10⁹ cells in the subject (such as the human patient). In certain embodiments, the pharmaceutical composition is administered at a dose resulting in about 6.5 to 7.5×10⁹ cells in the subject (such as the human patient). In certain embodiments, the pharmaceutical composition is administered at a dose resulting in about 7.0 to 8.0×10⁹ cells in the subject (such as the human patient). In certain embodiments, the pharmaceutical composition is administered at a dose resulting in about 7.5 to 8.5×10⁹ cells in the subject (such as the human patient). In certain embodiments, the pharmaceutical composition is administered at a dose resulting in about 8.0 to 9.0×10⁹ cells in the subject (such as the human patient). In certain embodiments, the pharmaceutical composition is administered at a dose resulting in about 8.5 to 9.5×10⁹ cells in the subject (such as the human patient). In certain embodiments, the pharmaceutical composition is administered at a dose resulting in about 9.0 to 10.0×10⁹ cells in the subject (such as the human patient).

In some cases, a subject method involves administering to a subject (such as a human patient) in need thereof an effective amount of a pharmaceutical composition described herein. In some embodiments, an “effective amount” of a pharmaceutical composition is an amount that, when administered to a subject (such as a human patient) in one or more doses, in monotherapy or in combination therapy, is effective to reduce symptoms of disease, such as a cancer, in the subject (such as the human patient) by at least about 20%, at least about 25%, at least about 30%, at least about 40%, at least about 50%, at least about 60%, at least about 70%, at least about 80%, at least about 2-fold, at least about 2.5-fold, at least about 3-fold, at least about 5-fold, at least about 10-fold, or greater than 10-fold, compared to the subject (such as the human patient) in the absence of treatment with the pharmaceutical composition.

In some embodiments, the kit and/or composition comprises a population of cells comprising γδ T cells described herein. In some embodiments, the kit may further comprise instruction(s) on methods of using the composition, such as uses described herein. The kits described herein may further include other materials desirable from a commercial and user standpoint, including other buffers, diluents, filters, needles, syringes, intravenous injectors or drips, and package inserts with instructions for performing any methods described herein.

In some embodiments, the kit and/or composition comprises a culture medium used in the methods provided herein, whether provided individually as components, in any combination, or as the culture medium admixed with cells. In some embodiments, the kit comprises reagents suitable for expanding the cells, such as media, cytokine, ITSEA, and human albumin serum. In some embodiments, the kit and/or composition comprises a culture medium used in the methods provided herein admixed with PBMCs, or a derivative thereof, e.g., intermediate compositions of the methods provided herein.

In some embodiments, the kit comprises one or more instruments to transduce a population of cells, such as PBMCs (or a derivative thereof) or γδ T cells, for the expression of a transgene, such as a chimeric antigen receptor (CAR) construct (such an instrument may be a syringe, pipette, forceps, and/or any such medically approved apparatus). In some embodiments, the kit comprises reagents or apparatuses for transduction of PBMCs (or a derivative thereof) or γδ T cells.

The components of the kits may be packaged either in aqueous media or in lyophilized form. The container means of the kits may include at least one vial, test tube, flask, bottle, syringe, or other container means, into which a component may be placed, and preferably, suitably aliquoted. Where there is more than one component in the kit, the kit also will generally contain a second, third, or other additional container into which the additional components may be separately placed. However, various combinations of components may be comprised in a vial. The kits of the present invention also will typically include a means for containing the population of cells, such as PBMCs (or a derivative thereof) or γδ T cells, and any other reagent containers in close confinement for commercial sale. Such containers may include injection or blow molded plastic containers into which the desired vials are retained, for example.

In some embodiments, the kits may further comprise instruction(s) on methods of manufacturing or using the populations of cells or the composition thereof, such as methods of manufacturing or uses described herein.

### V. Methods of Use

In one aspect is provided a method of treating a disease in an individual, wherein the method comprises: obtaining an isolated pool of mixed immune cells depleted of αβ T cells, such as isolated PCMCs depleted of αβ T cells, or a derivative thereof, from a donor individual; producing a population of cells comprising γδ T cells using a method described herein; and administering the population of cells comprising γδ T cells to the individual. In some embodiments, the donor individual is not the individual (such as in an allogeneic cell therapy setting). In some embodiments, the donor individual is the individual (such as in an autologous cell therapy setting).

In some embodiments, the disease is selected from any one of a cancer, an infectious disease, an autoimmune disease, or another disease wherein immune function is implicated (e.g., endometriosis). In some embodiments, the disease is a cancer.

The methods are applicable to cancers of all stages, including early-stage cancer, non-metastatic cancer, primary cancer, advanced cancer, locally advanced cancer, metastatic cancer, or cancer in remission. In some embodiments, the cancer has been refractory to prior therapy. In some embodiments, the cancer can be a solid tumor malignancy. In some embodiments, the cancer can be a liquid cancer. In some embodiments, the malignancy can include, but is not limited to, any one of: Acute Lymphoblastic Leukemia (ALL), Acute Myeloid Leukemia (AML), Adrenocortical Carcinoma, Kaposi Sarcoma (Soft Tissue Sarcoma), AIDS-Related Lymphoma (Lymphoma), Primary CNS Lymphoma (Lymphoma), Anal Cancer, Appendix Cancer, Astrocytomas, Atypical Teratoid/Rhabdoid Tumor, Basal Cell Carcinoma of the Skin, Bile Duct Cancer, Bladder Cancer, Bone Cancer (includes Ewing Sarcoma and Osteosarcoma and Malignant Fibrous Histiocytoma), Brain Tumors, Breast Cancer, Bronchial Tumors, Burkitt Lymphoma, Carcinoid Tumor, Carcinoma, Cardiac Tumors, Atypical Teratoid/Rhabdoid Tumor, Medulloblastoma, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma, Ductal Carcinoma In Situ (DCIS), Endometrial Cancer, Ependymoma, Esophageal Cancer, Esthesioneuroblastoma, Ewing Sarcoma, Extracranial Germ Cell Tumor, Extragonadal Germ Cell Tumor, Intraocular Melanoma, Retinoblastoma, Fallopian Tube Cancer, Fibrous Histiocytoma of Bone, Osteosarcoma, Gallbladder Cancer, Gastric Cancer, Gastrointestinal Carcinoid Tumor, Gastrointestinal Stromal Tumors (GIST) (Soft Tissue Sarcoma), Germ Cell Tumors, Central Nervous System Germ Cell Tumors, Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Hepatocellular Cancer, Histiocytosis (Langerhans Cell), Hodgkin Lymphoma, Hypopharyngeal Cancer, Islet Cell Tumors, Pancreatic Neuroendocrine Tumors, Kaposi Sarcoma (Soft Tissue Sarcoma), Renal Cell Cancer, Langerhans Cell Histiocytosis, Laryngeal Cancer, Leukemia, Lip and Oral Cavity Cancer, Liver Cancer, Lung Cancer (Non-Small Cell, Small Cell, Pleuropulmonary Blastoma, and Tracheobronchial Tumor), Lymphoma, Male Breast Cancer, Malignant Fibrous Histiocytoma of Bone and Osteosarcoma, Merkel Cell Carcinoma, Mesothelioma, Metastatic Cancer, Metastatic Squamous Neck Cancer with Occult Primary, Midline Tract Carcinoma With NUT Gene Changes, Oropharyngeal Cancer, Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Chronic Myelogenous Leukemia (CML), Acute Myeloid Leukemia (AML), Chronic Myeloproliferative Neoplasms, acute B lymphoblastic leukemia (B-ALL), diffuse large B cell lymphoma (DLBCL), Follicular Lymphoma, Blastic Plasmacytoid Dendritic Cell Neoplasm (BPDCN), Systemic Mastocytosis, Nasal Cavity and Paranasal Sinus Cancer, Nasopharyngeal Cancer, Neuroblastoma, Non-Hodgkin Lymphoma, Non-Small Cell Lung Cancer, Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Primary Central Nervous System (CNS) Lymphoma, Primary Peritoneal Cancer, Prostate Cancer, Recurrent Cancer, Rhabdomyosarcoma, Salivary Gland Cancer, Vascular Tumors, Small Intestine Cancer, Soft Tissue Sarcoma, T-Cell Lymphoma, Thymoma and Thymic Carcinoma, Transitional Cell Cancer of the Renal Pelvis and Ureter, Vaginal Cancer, Vulvar Cancer, or Wilms Tumor, etc.

In some embodiments, the populations of cells comprising γδ T cells produced using the methods described herein further comprise a chimeric antigen receptor (CAR). In some embodiments, the CAR comprises an antigen binding domain, a hinge domain, a transmembrane domain, and an intracellular signaling domain comprising a costimulatory domain and/or a primary signaling domain. In some embodiments, the populations of cells comprising γδ T cells produced using the methods described herein further comprise an engineered T cell receptor (TCR). In some embodiments, the engineered TCR comprises an engineered antigen binding domain. In some embodiments, said CAR or engineered TCR antigen binding domain binds to a tumor antigen selected from a group consisting of: mesothelin, EGFRVIII, TSHR, CD19, CD123, CD22, CD30, CD171, CS-1, CLL-1, CD33, GD2, GD3, BCMA, TROP-2, Tn Ag, prostate specific membrane antigen (PSMA), ROR1, FLT3, FAP, TAG72, CD38, CD44v6, CEA, EPCAM, B7H3, KIT, IL-13Ra2, interleukin-11 receptor a (IL-11Ra), PSCA, PRSS21, VEGFR2, LewisY, CD24, platelet-derived growth factor receptor-beta (PDGFR-beta), SSEA-4, CD20, Folate receptor alpha (FRa), ERBB2 (Her2/neu), HER3, MUC1, epidermal growth factor receptor (EGFR), TGF-β, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, CLDN18, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD5, CD7, Nectin-4, PD-L1, HLA-E, HLA-G, MIC A/B, AKR1C3, or ULBP1-6.

In some embodiments, the transmembrane domain of the CAR can be selected from the group consisting of: CD8a, CD4, CD28, CD45, NKG2D, NKp30, NKp44, NKp46, DNAMI, 2B4, PD1, and CD152. In some embodiments, the intracellular signaling domain of the CAR can be selected from the group consisting of: CD3, CD27, CD28, DNAM1, 2B4, DAP10, DAP12, CD54 (ICAM), CD134 (OX40), CD137(41BB), CD 152 (CTLA4), CD273 (PD-L2), CD274 (PD-L1), and CD278 (ICOS). In some embodiments, the hinge domain of the CAR can be selected from the group consisting of: CD8, CD4, CD28, IgG, and CD34.

In some embodiments, the disease is an infectious disease, such as a bacterial, viral, fungal, or parasitic infectious disease. In some embodiments, the infectious disease is chronic.

Examples of bacterial infections include, but are not limited to: Mycobacterium (e.g., M. tuberculosis, M. leprae), Brucella (e.g., B. abortus), Corynebacteria (e.g., C. diptheriae), Vibrio (e.g., V. cholerae), Bordetella (e.g. B. pertussis), Clostridium (e.g., C. tetani, C. botulinum, and C. difficile), Yersinia (e.g., Y. pestis, Y. enterocolitica), Neisseria (e.g., N. gonorrhoeae, N. meningitidis), Treponema (e.g., T. pallidum), Chlamydophila (e.g., C. pneumoniae and C. trachomatis), Aeromonas (e.g., A. caviae, A. hydrophila, and A. veronii), Salmonella (e.g., S. enteritis), Legionella (e.g., L. pneumophila), Bacillus (e.g., B. anthracis and B. cereus), Leptospira (e.g., Leptospira sp.), Klebsiella (e.g., K. pneumonia), Streptococcus (e.g., S. pneumoniae), Listeria (e.g., L. monocytogenes), Pseudomonas (e.e., P. aeruginosa), Staphylococcus (e.g., S. aureus), or Escherichia (e.g., E. coli).

Examples of mammalian viral infections include, but are not limited to: infections caused by DNA Viruses (e.g., Herpes Viruses such as Herpes Simplex viruses, Epstein-Barr virus, Cytomegalovirus; Pox viruses such as Variola (small pox) virus; Hepadnaviruses (e.g., Hepatitis B virus; Papilloma viruses; Adenoviruses); RNA Viruses (e.g., HIV I, II; HTLV I, II; Poliovirus; Hepatitis A; Orthomyxoviruses (e.g., Influenza viruses); Paramyxoviruses (e.g., Measles virus); Rabies virus; Hepatitis C); Coronavirus (causes Severe Acute Respiratory Syndrome (SARS)); Rhinovirus, Respiratory Syncytial Virus, Norovirus, West Nile Virus, Yellow Fever, Rift Valley Virus, Lassa Fever Virus, Ebola Virus, and Lymphocytic Choriomeningitis Virus.

Examples of fungal infections include, but are not limited to: infections caused by Candida (e.g., C. albicans, C. glabrata, C. guilliermondii, C. krusei, C. lusitaniae, C. pseudotropicalis, C. rugosa, C. stellatoidea, C. parapsilosis, C. tropicalis, and C. auris), Cryptococcus (e.g., C. neoformans), Aspergillus (e.g., A. fumigatus, A. flavus, A. niger, A. terreus, and A. nidulans), Coccidioides (e.g., C. immitis and C. posadasii; also known as Valley Fever), Histoplasma (e.g., H. capsulatum), Blastomyces (e.g., B. dermatitidis), Pneumocystis (e.g., P. jirovecii), Penicillium (e.g., P. marneffei), Mucorales (e.g., Rhizopus oryzae, R. arrhizus, R. rhizopodiformis, Rhizomucor pusillus, Absidia oryzae, A. corymbifera, A. ramosa, and Mucor circinelloides), Sporothrix (e.g., S. schenckii), Fusarium (e.g., F. solani, F. oxysporum, F. moniliforme, F. dimerum, F. chlamydosporum, and F. anthophilum), Trichosporon (e.g., T. beigelii and T. capitatum), Geotrichum (e.g., G. candidum), Rhodotorula (e.g., R. rubra), or Dematiaceous molds or pheohyphomycetes (e.g., Pseudallescheria boydii (Scedosporium apiospermum), Bipolaris, Alternaria, and Scedosporium prolificans).

Examples of parasitic infections include, but are not limited to: infections caused by Acanthamoeba (such as Acanthamoeba keratitis, Granulomatous Amebic Encephalitis, or Disseminated infection), African Trypanosomiasis or sleeping sickness (e.g., Trypanosoma brucei), Echinococcosis (e.g., Echinococcus granulosus or E. multiocularis), Amebiasis (e.g., Entamoeba histolytica), Chagas disease (e.g., Trypanosoma cruzi), Hookworm (such as Ancylostoma brazilense, A. caninum, A. ceylanicum, and Uncinaria stenocephala), Angiostrongyliasis (e.g., Angiostrongylus cantonensis), Anisakiasis (e.g., Pseudoterranova decipiens), Ascariasis (e.g., Ascaris lumbricoides or Ascaris suum), Babesiosis (e.g., Babesia microti), Balantidiasis (e.g., Balantidium coli), Balamuthia (e.g., Balamuthia mandrillaris), Baylisascariasis (e.g., Baylisascaris procyonis), Bed Bugs, Schistosomiasis (such as Schistosoma mansoni, S. haematobium, or S. japonicum), Blastocystis hominis infection, Body Lice infestation, Capillariasis (e.g., Capillaria hepatica or Capillaria philippinensis), Cercarial Dermatitis (e.g., Austrobilharzia variglandis), nonpathogenic intestinal protozoa (such as Chilomastix mesnili, Endolimax nana, Entamoeba coli, Entamoeba dispar, Entamoeba hartmanni, Entamoeba polecki, and Iodamoeba buetschlii), Clonorchiasis or liver flukes (e.g., Clonorchis sinensis, Opisthorchi viverrinis, or O. felineus), Pubic Lice, Cryptosporidiosis (e.g., Cryptosporidium), Cyclosporiasis (e.g., Cyclospora cayetanensis), Cysticercosis (e.g., Taenia solium), Cystoisosporiasis or Isospora infection (e.g., Cystoisospora belli), Dientamoeba fragilis infection, Diphyllobothriasis (e.g., Diphyllobothrium latum), Dipylidium caninum infection or dog/cat tapeworm, Dirofilariasis (e.g., Dirofilaria immitis), Dracunculiasis or Guinea Worm disease (Dracunculus medinensis), Elephantiasis or filariasis (e.g., Wuchereria bancrofti or Brugia malayi), Enterobiasis or pinworm (Enterobius vermicularis), Fascioliasis or common liver fluke (e.g., Fasciola hepatica or F. gigantica), Fasciolopsiasis (e.g., Fasciolopsis buski), Giardiasis (e.g., Giardia intestinalis), Gnathostomiasis (e.g., Gnathostoma spinigerum), Heterophyiasis (e.g., Heterophyes heterophyes), Hymenolepiasis (e.g., Hymenolepis nana), common foodborne parasites (including protozoa such as Cryptosporidium spp., Giardia intestinalis, Cyclospora cayetanensis, and Toxoplasma gondii; roundworms such as Trichinella spp. and Anisakis spp.; and tapeworms such as Diphyllobothrium spp. and Taenia spp.), Leishmaniasis (e.g., Leishmania promastigotes), Loiasis or African eye worm (e.g., Loa loa), Malaria (e.g., Plasmodium spp.), Microsporidiosis (e.g., Enterocytozoon bieneusi, E. cuniculi, E. hellem, E. intestinalis, Anncaliia algerae, A. connori, A. vesicularum, Microsporidium ceylonensis, M. africanum, Trachipleistophora hominis, T. anthropophthera, Nosema ocularum, Pleistophora ronneafiei, Vittaforma corneae, or Tubulinosema acridophagus), Mite infestation (e.g., Sarcoptes scabiei var. hominis), Myiasis or fly maggot infection (e.g., Dermatobia hominis), Naegleria Infection (e.g., Naegleria fowleri), Toxocariasis (e.g., Toxocara canis or Toxocara cati), Onchocerciasis or river blindness (e.g., Onchocerca volvulus), Paragonimiasis or lung fluke (e.g., Paragonimus westermani), Pneumocystis jirovecii pneumonia, Sappinia amoeba (e.g., Sappinia diploidea or Sappinia pedata), Sarcocystosis (e.g., Sarcocystis sp.), Strongyloidiasis (e.g., Strongyloides stercoralis), Trichinellosis (e.g., Trichinella sp.), Trichomoniasis (e.g., Trichomonas vaginalis), Trichuriasis or whipworm (e.g., Trichuris trichiura), etc.

In some embodiments, the disease is an autoimmune disorder. Examples of autoimmune disorders include, but are not limited to: graft-versus-host disease, autoimmune polyendocrinopathy syndromes, Type I diabetes mellitus (TIDM), autoimmune gastritis, autoimmune uveoretinitis, autoimmune vasculitis, colitis, thyroiditis, Addison's disease, agammaglobulinemia, alopecia areata, amyloidosis, ankylosing spondylitis, anti-GBM/anti-TBM nephritis, antiphospholipid syndrome, autoimmune hepatitis, autoimmune inner ear disease, axonal & neuronal neuropathy, Behqet's disease, bullous pemphigold, Castleman disease, celiac disease, Chagas disease, chronic inflammatory demyelinating polyneuropathy(CIDP), chronic recurrent multifocal osteomyelitis, Churg-Strauss, cicatrical pemphigold/benign mucosal pemphigold, Cogan's syndrome, cold agglutinin disease, congenital heart block, coxsackie myocarditis, CREST syndrome, Crohn's disease, dermatitis herpetiformis, dermatomyositis, Devic's disease, discoid lupus, Dressler's syndrome, endometriosis, eosinophilic esophagitis, eosinophilic fasciitis, erythema nodosum, essential mixed cryoglobulinemia, Evans syndrome, fibromyalgia, fibrosing alveolitits, giant cell arteritis (temporal arteritis), giant cell myocarditis, glomerulonephritis, Goodpasture's syndrome, granulomatosis with polyangitis, Grave's disease, Guillian-Barre syndrome, Hashimoto's thyroiditis, hemolytic anemia, Henoch-Schonnlien purpura, herpes gestationis or pemphigold gestationis, hypogammaglobulinemia, IgA nephropathy, IgG4-related sclerosing disease, inclusion body myositis, interstitial cystitis, juvenile arthritis, juvenile diabetes (Type1diabetes), juvenile myositis, Kawasaki disease, Lambert-Eaton syndrome, leukocyteclastic vasculitis, lichen planus, lichen sclerosis, ligneous conjunctivitis, linear IgA disease, lupus, Lyme disease, chronic Meniere's disease, microscopic polyanglitis, mixed connective tissue disease, Mooren's ulcer, Mucha-Habermann disease, multiple sclerosis, myasthenia gravis, myositis, narcolepsy, neuromyelitis opica, neutropenia, ocular cicatrical pemphigold, optic neuritis, palindromic rheumatism, PANDAS (pediatric autoimmune neuropsychiatric disorders associated with streptococcus), paraneoplastic cerebellar degeneration, paroxysmal nocturnal hemoglobinuria, Parry Romber syndrome, pars planitis (peripheral uveitis), Parsonnage-Turner syndrome, pemphigus, peripheral neuropathy, perivenous encephalomyelitis, pernicious anemia, POEMS syndrome, polyarteritis nodosa, polymyalgia rheumatic, polymyositis, postmycoardial infarction syndrome, postpericadiotomy syndrome, primary biliary cirrhosis, primary sclerosing cholangitis, progesterone dermatitis, psoriasis, psoriatic arthritis, pure red cell aplasia, pyoderma gangrenosum, Raynaud's phenomenon, reactive arthritis, reflex sympathetic dystrophy, Reiter's syndrome, relapsing polychondritis, restless leg syndrome, retroperitoneal fibrosis, rheumatic fever, rheumatoid arthritis, sarcoidosis, Schmidt syndrome, scleritis, scleroderma, Sjogren's syndrome, sperm and testicular autoimmunity, stiff person syndrome, subacute bacterial endocarditis, Susac's syndrome, sympathetic ophtalmia, Takayasu's arteritis, temporal arteritis/giant cell arteritis, thrombocytopenia purpura, Tolosa-Hunt syndrome, transverse myelitis, ulcerative colitis, undifferentiated connective tissue disease, uveitis, vasculitis, vitiligo, and Wegner's granulomatosis (granulomatosis with polyangitis).

### VI. EXAMPLES

The examples below are intended to be purely exemplary of the invention and should therefore not be considered to limit the invention in any way. The following examples and detailed description are offered by way of illustration and not by way of limitation. For the embodiments in which details of the experimental methods are not described, such methods are carried out according to conventional conditions such as those described in Sambrook et al.

Molecular Cloning: A Laboratory Manual (New York: Cold Spring Harbor Laboratory Press, 1989), or as suggested by the manufacturers.

### A. Example 1: Expansion of γδ T Cells in Vitro

Described herein is the novel method used to generate a pure γδ T cell population in vitro. To acquire the starting cell population, human peripheral blood cells (hPBMCs) were collected by apheresis. These harvested cells then were stained with an anti-TCR α/β antibody (Miltenyi Biotec) followed by the removal of the antibody-conjugated αβ TCR+ T cells by the CliniMACS plus system (Miltenyi Biotec). The remaining cells were cryopreserved for future γδ T cell expansion.

To expand γδ T cells, αβ TCR-depleted cells were thawed and cultured in medium containing the following components for 14 days:
Basal medium: NK MACS medium (Miltenyi Biotec)
IL-15 (10 ng/ml)
Human AB serum (1%)
ITSEA (outlined in Table 1 below).

TABLE 1
Components of ITSEA and the final (i.e., working) concentrations
of each component for γδ T cell expansion in vitro.

| ITSEA components | Final conc (mg/L) in cell medium |
|---|---|
| Human insulin | 20 |
| Human transferrin | 11 |
| Sodium selenite | 0.0134 |
| Ethanolamine | 4 |
| Human albumin | 400 |

Medium was either refreshed once on Day 7 or twice on Day 7 and Day 11 (or 12) by replacing ~80% old medium with an equal volume of fresh medium. The γδ T cells were cultured in vitro for approximately 14 days. During cell culture, recombinant human IL-15 was added once every 2-3 days to the cell culture medium. Fresh ITSEA was added to fresh cell culture medium when the medium was refreshed on Day 7 or on Days 7 and 11. Cells were harvested and cryopreserved on Day 14.

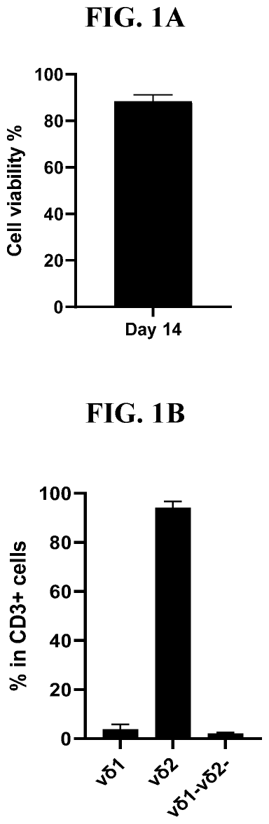

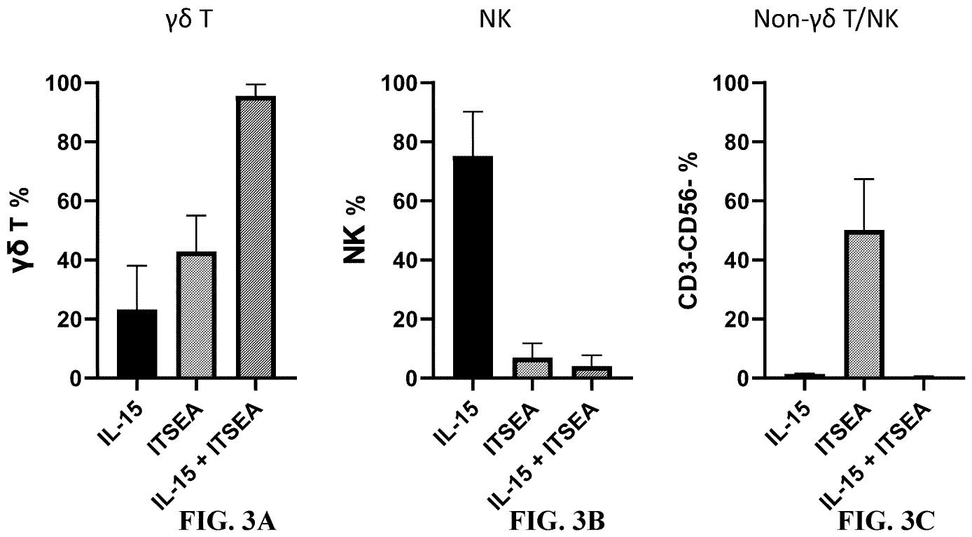

Results showed that culturing the αβ TCR-depleted cells for 14 days in a basal medium plus IL-15 and ITSEA led to approximately >2000-fold expansion of γδ T cells (FIG. 2A), of which approximately 90% were alive (FIG. 1A), with a purity at approximately 95% across two separate trial runs (FIG. 3A). As shown in FIG. 1B, the majority of the CD3+ T cells are vδ2 γδ T cells (>90%) and approximately 5% are vδ1 γδ T cells. Not only is this protocol effective and efficient, but it is also consistent, which is crucial for scaling to meet the demands for downstream applications. Such findings are significant in view of expansion achieved with comparable methods, e.g., Dokouhaki et al. (2010) Cancer Lett 297(1): 126-136; Van Acker et al. (2016) J Hematol Oncol 9(1): 101; Li et al. (2010) J Immunother 33 (3): 287-296; Salot et al. (2007) J Immunol Methods 326(1-2): 63-75; and Kondo et al. (2008) Cytotherapy 10(8): 842-856.

The addition of IL-15 and ITSEA, alone or in combination, to a basal medium in the presence of 1% human AB serum for stimulating γδ T cell expansion was tested. As shown in FIGS. 2A-C, each single component was not able to expand γδ T cells significantly or generate pure γδ T cells. Upon combining IL-15 and ITSEA in the cell culture medium described above, γδ T cell expansion dramatically increased, and high γδ T purity was achieved in the final cell product. As seen in FIG. 2A, γδ T cells co-treated with IL-15 and ITSEA showed an increase in expansion from about 300-fold with IL-15 alone to about 2500-fold with co-treatment. FIG. 2B shows a marked reduction in the expansion of NK cells in the sample when co-treated with IL-15 and ITSEA (approximately 10-fold) compared to IL-15 treatment alone (approximately 60-fold). FIG. 2C shows a lack of expansion of CD3-CD56-cells (non-γδ T or NK cells).

FIG. 3A shows that approximately 95% of the cells produced in culture with IL-15 and ITSEA treatment are γδ T cells. FIG. 3B shows that NK cells predominate in the cell mixture when cultured with IL-15 alone, whereas NK cells make up about 5% of the overall cell population when cultured with IL-15 and ITSEA. FIG. 3C shows that non-γδ T cells and non-NK cells make up a significant percentage of the cell population when cultured with ITSEA alone but are not distinguishably present when cultured with IL-15 and ITSEA in combination. These results demonstrate the high γδ T cell purity for the IL-15 and ITSEA co-treatment condition.

These results show that this protocol is effective at rapid generation of a pure γδ T cell population from the starting αβ TCR-depleted cell population, which is consistently achieved across multiple trials.

### B. Example 2: Cellular Phenotype of γδ T Cells Expanded In Vitro

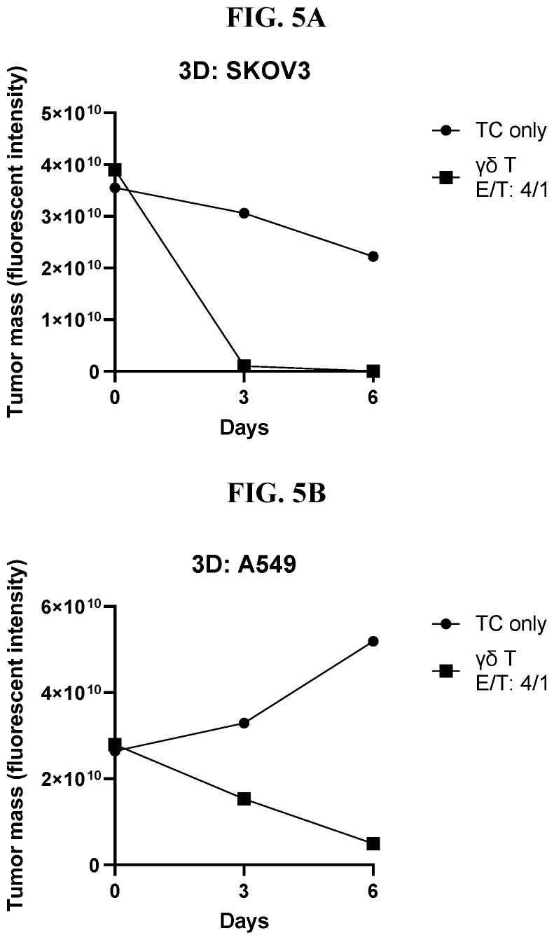

The γδ T cells were also tested for in vitro antitumor activities in 2D (FIGS. 4A-5B) and 3D (FIGS. 5A-5B) conditions. In brief, in the 2D assays, luciferase expressing SKOV3 and A549 cancer cell lines were pretreated with vehicle or zoledronate (Zol) to boost antigen expression in tumor cells. These cancer cell lines were then co-cultured with γδ T cells at an Effector to Target (E/T) ratio of 4/1 in the presence of recombinant human IL-15(rhIL-15 ) at 10 ng/ml. In the 3D assays, fluorescent protein-tagged SKOV3 and A549 tumor cells were plated in 3D culture to allow tumorsphere formation. Two days later, γδ T cells were added at E/T=4/1 with rhIL-15 at 10 ng/mL.

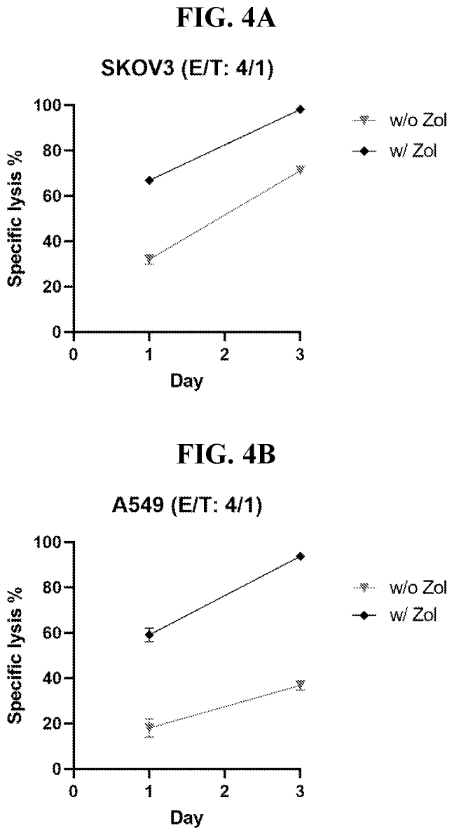

In the 2D assays, cytotoxicity (Specific lysis %) was determined at Day 1 and Day 3 based on the loss of the luciferase signals. γδ T cells induced SKOV3 (FIG. 4A) and A549 (FIG. 4B) tumor cell death in a time-dependent manner. Pretreatment of tumor cells with zoledronate dramatically boosted γδ T cell-mediated tumor cell killing to nearly 100% on Day 3.

In 3D assays, fluorescent intensities signifying tumor mass were determined on Days 3 and 6. Results suggest that in the 3D condition, the γδ T cells showed robust antitumor activity and completely destroyed SKOV3 (FIG. 5A) and A549 (FIG. 5B) tumorspheres even in the absence of zoledronate pretreatment, as shown by the nearly complete loss of tumor mass in FIGS. 5A-5B.

### C. Example 3: Use of γδ T Cells for CAR-T Cell Therapy in an In Vitro Cancer Cell Co-Culture Model

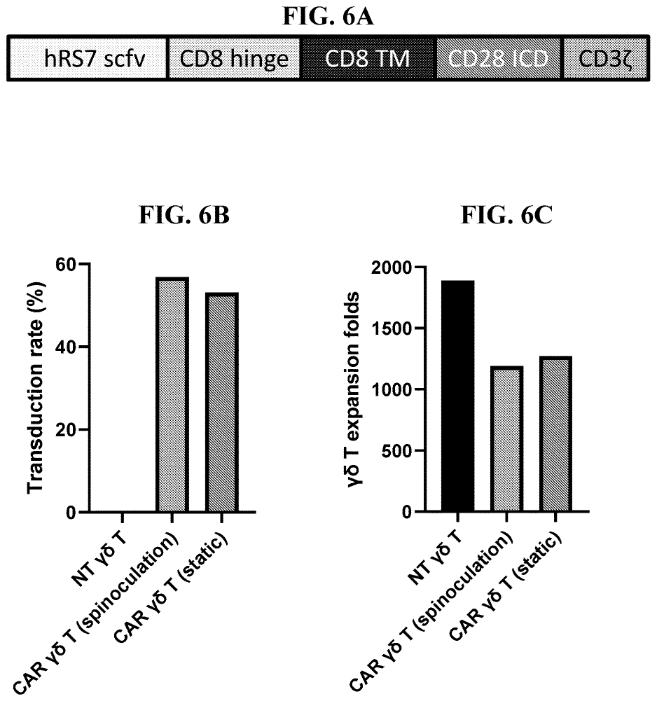

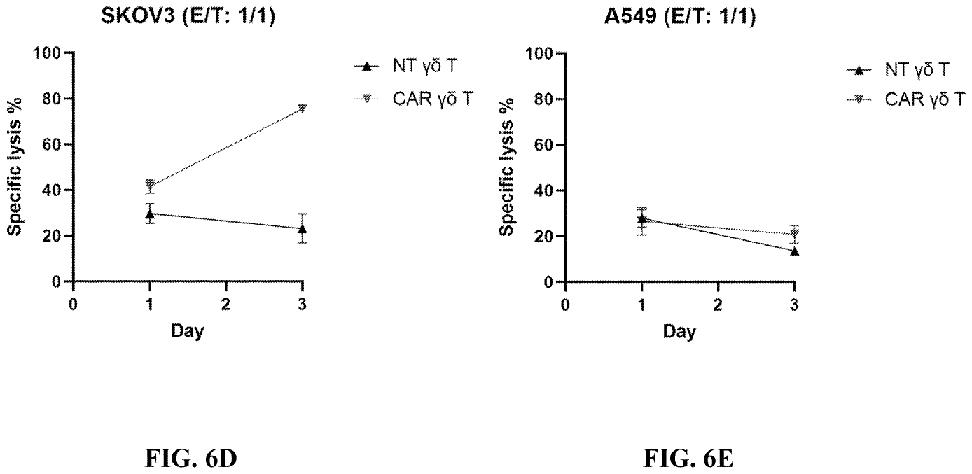

γδ T cells were generated as described in Example 1 above. Cells were cultured for 4 days before viral transduction of a TROP-2-targeting CAR construct as shown by the schematic in FIG. 6A. The light and heavy chains coding sequences of a TROP2 monoclonal antibody (hRS7) were converted to a single-chain variable fragment (scFv) and inserted in a retroviral vector upstream to the remaining CAR domains: CD8 hinge, CD8 transmembrane domain (TM), intracellular CD28 costimulatory domain (CD28 ICD), and intracellular CD3 activation domain (CD3ξ). The CAR-expressing retroviral vector was transfected in a retroviral packaging cell line to produce the viral medium, which was then used to transduce the αβ TCR-depleted cells on Day 4 of in vitro cell culture.

Transduction Method 1: On Day 4, cells were harvested and retroviral transduction was carried out by spinoculation. The viral medium was loaded to Retronectin-coated cell culture bags. The bags were centrifuged at 2000 g for 2 hours at 32° C. Next, the virus-containing medium was removed. Cells were added in the γδ T cell medium (described in Example 1) to the virus-coated bags. The bags were then centrifuged at 1000 g for 10 min at 32° C. followed by incubation at 37° C. for 2 days.

Transduction Method 2: Retroviral transduction was also performed using static incubation. The viral medium was loaded to Retronectin-coated cell culture bags. Without centrifugation, cells were added in the γδ T cell medium (described in Example 1) to the virus-loaded bags. The bags were incubated at 37° C. followed by incubation at 37° C. for 2 days.

On Day 6, cells were harvested and replated in the γδ T cell medium (described in Example 1) for cell expansion. Medium was refreshed once on Day 11 and contained fresh ITSEA. Cells were further cultured for 3 more days for a total of 14 days of culture. 10 ng/ml rhIL-15 was added every 2-3 days. Cells were harvested and cryopreserved on Day 14.

Tumor killing assay: γδ CAR-T cells and non-transduced control γδ T cells were co-cultured with SKOV3 or A549 cancer cells in order to assess the targeted cytotoxicity of the γδ CAR-T cells. SKOV3 cancer cells expressed the TROP-2 antigen, to which the CAR was engineered to recognize. In contrast, the A549 cancer cells did not express the TROP-2 antigen. Both SKOV3 and A549 cell lines expressed luciferase, which was used to calculate specific lysis of the target cancer cells by the γδ CAR-T cells.

As shown in FIG. 6B, retroviral transduction results in >50% transduction rate by either transduction approach. Transduced γδ T cells, regardless of the method of transduction, reached >1000-fold expansion by Day 14 (FIG. 6C). Upon co-culturing the γδ CAR-T cells or the non-transduced (NT) γδ T cells with SKOV3 or A549 cancer cells (as described in Example 2 above), the γδ CAR-T cells, but not NT γδ T cells, displayed increased cancer cell killing of specifically of TROP2-positive (TROP2+) SKOV3 tumor cells (FIG. 6D) but not of TROP2-negative (TROP2-) A549 cells (FIG. 6E), in a time-dependent manner, thereby demonstrating successful targeting and cytotoxic activity against specific antigen (i.e., TROP-2)-expressing cancers.

The results described above support that the invention provides a novel feeder-cell-and an antibody-free approach (such as a γδ TCR-stimulating antibody-free approach) for expanding γδ T cells, which can further include the process of engineering γδ T cell, e.g., to generate γδ CAR-T cells. This invention eliminates the extra manufacturing steps for feeder cells and activating antibodies, the QA/QC steps for checking feeder cell contents in the final products, and the safety concerns of introducing residual feeder cells to human patients. The end-product is highly pure γδ T cells, thereby eliminating the steps for γδ T cell enrichment or isolation after successfully expanding the cells. No αβ T cell contamination was identified in the final cell product, which further minimizes the risk of a potential patient developing GvHD if administrated as an allogeneic cell therapy.

The γδ T cells demonstrated potent antitumor activities in 2D and 3D cell culture models. Moreover, the γδ T cell manufacturing protocol was compatible with cell engineering methods, exemplified by retroviral transduction. γδ CAR-T cells generated by the γδ T cell protocol showed significantly increased tumor cell-killing capability compared to unmodified γδ T cells.

Together, these results show that the invention may be used to produce highly pure and active γδ T cells and γδ CAR-T cells that can be used for further downstream applications, such as in allogeneic or autologous cell therapies for patients with various diseases.

All references mentioned in the present invention are incorporated herein by reference as if each of those references has been incorporated by reference individually. Although the description referred to particular embodiments, it will be clear to a person skilled in the art that the present invention may be practiced with variation of these specific details. Hence this invention should not be construed as limited to the embodiments set forth herein.

What is claimed is:

1. A method for producing a population of cells comprising γδ T cells, the method comprising:
culturing an isolated pool of mixed immune cells depleted of αβ T cells, or a derivative thereof, in a culture medium comprising:
(a) a basal medium;
(b) a cytokine; and
(c) one or more of the following:
(i) a metabolic polypeptide;
(ii) an iron source;
(iii) an antioxidant enzyme cofactor;
(iv) a lipid precursor; and
(v) a carrier protein; and
harvesting the population of cells comprising γδ T cells.

2. The method of claim 1, wherein the population of cells comprising γδ T cells has a γδ T cell purity of at least about 85%. The method of claim 1 or 2, wherein the population of cells comprising γδ T cells has a γδ T cell purity of at least about 95%.

4. The method of any one of claims 1-3, wherein the population of cells comprising γδ T cells comprises vδ2 γδ T cells, and, optionally, vδ1 γδ T cells.

5. The method of any one of claims 1-4, wherein the population of cells comprising γδ T cells has a vδ2 γδ T cell purity of at least about 70% relative to total γδ T cells in the population.

6. The method of any one of claims 1-5, wherein the population of cells comprising γδ T cells has a vδ2 γδ T cell purity of at least about 95% relative to total γδ T cells in the population.

7. The method of any one of claims 1-6, wherein the population of cells comprising γδ T cells comprises less than about 15% of NK cells.

8. The method of any one of claims 1-7, wherein the population of cells comprising γδ T cells comprises less than about 2.5% of NK cells.

9. The method of any one of claims 1-8, wherein the basal culture medium is NK MACS medium.

10. The method of any one of claims 1-9, wherein the cytokine is one or more of IL-15, IL-2, IL-7, SCF, TPO, FLT-3L, BMP4, VEGF, or bFGF.

11. The method of claim 10, wherein the cytokine is IL-15.

12. The method of any one of claims 1-11, wherein the cytokine is added to the culture medium in an amount of about 100 pg/mL to about 500 ng/mL.

13. The method of any one of claims 1-12, wherein the metabolic polypeptide is an insulin or analog thereof.

14. The method of claim 13, wherein the insulin is a human insulin.

15. The method of any one of claims 1-14, wherein the metabolic polypeptide is added to the culture medium in an amount of about 1 mg/L to about 50 mg/L.

16. The method of any one of claims 1-15, wherein the iron source is a transferrin.

17. The method of claim 16, wherein the transferrin is a human transferrin.

18. The method of any one of claims 1-17, wherein the iron source is added to the culture medium in an amount of about 1 mg/L to about 50 mg/L.

19. The method of any one of claims 1-18, wherein the antioxidant enzyme cofactor is a selenium compound.

20. The method of claim 19, wherein the selenium compound is sodium selenite.

21. The method of any one of claims 1-20, wherein the antioxidant enzyme cofactor is added to the culture medium in an amount of about 0.001 mg/L to about 0.1 mg/L.

22. The method of any one of claims 1-21, wherein the lipid precursor is ethanolamine.

23. The method of any one of claims 1-22, wherein the lipid precursor is added to the culture medium in an amount of about 1 mg/L to about 10 mg/L.

24. The method of any one of claims 1-23, wherein the carrier protein is an albumin.

25. The method of claim 24, wherein the albumin is a human albumin.

26. The method of claim 25, wherein the human albumin is human serum albumin.

27. The method of any one of claims 1-26, wherein the carrier protein is added to the culture medium in an amount of about 100 mg/L to about 5,000 mg/L.

28. The method of any one of claims 1-27, wherein the culture medium comprises two or more of:
(i) the metabolic polypeptide;
(ii) the iron source;
(iii) the antioxidant enzyme cofactor;
(iv) the lipid precursor; and
(v) the carrier protein.

29. The method of any one of claims 1-28, wherein the culture medium comprises three or more of:
(i) the metabolic polypeptide;
(ii) the iron source;
(iii) the antioxidant enzyme cofactor;
(iv) the lipid precursor; and
(v) the carrier protein.

30. The method of any one of claims 1-29, wherein the culture medium comprises four or more of:
(i) the metabolic polypeptide;
(ii) the iron source;
(iii) the antioxidant enzyme cofactor;
(iv) the lipid precursor; and
(v) the carrier protein.

31. The method of any one of claims 1-30, wherein the culture medium comprises:
(i) the metabolic polypeptide;
(ii) the iron source;
(iii) the antioxidant enzyme cofactor;
(iv) the lipid precursor; and
(v) the carrier protein.

32. The method of claim 31, wherein the culture medium comprises ITSEA.

33. The method of any one of claims 1-32, wherein the culture medium further comprises a serum.

34. The method of claim 33, wherein the serum is an AB serotype (AB) serum.

35. The method of claim 33 or 34, wherein the AB serum is a human AB serum.

36. The method of any one of claims 33-35, wherein the serum is added to the culture medium in an amount of about 0.1% (v/v) to about 10% (v/v).

37. The method of any one of claims 1-36, wherein the culturing is from about 0 to about 14 days.

38. The method of any one of claims 1-37, further comprising cryopreserving the harvested population of cells comprising γδ T cells.

39. The method of claim any one of claims 1-38, wherein the isolated pool of mixed immune cells depleted of αβ T cells are derived from Peripheral Blood Mononuclear Cells (PBMCs).

40. The method of any one of claims 1-38, wherein the isolated pool of mixed immune cells depleted of αβ T cells are derived from a stem cell bank or cell line.

41. The method of any one of claims 1-40, further comprising performing a step of αβ T cell depletion on the isolated pool of mixed immune cells.

42. The method of claim 41, wherein the step of αβ T cell depletion comprises an antibody-based depletion.

43. The method of claim 41 or 42, wherein the step of αβ T cell depletion comprises (a) subjecting the isolated pool of mixed immune cells to an anti-TCR α/β antibody; and (b) separating the antibody-conjugated αβ TCR+ T cells from other cells in the isolated pool of mixed immune cells to obtain an αβ T cell depleted population of cells.

44. The method of claim 43, wherein the separating is performed by a cell sorting technique.

45. The method of any one of claims 41-44, further comprising cryopreserving cells obtained from the step of αβ T cell depletion on the isolated pool of mixed immune cells prior to performing the culturing isolated pool of mixed immune cells, or the derivative thereof, in the culture medium.

46. The method of any one of claims 1-45, further comprising isolating the pool of mixed immune cells from a donor.

47. The method of claim 46, wherein the donor is not an intended recipient.

48. The method of claim 46, wherein the donor is an intended recipient.

49. The method of any one of claims 46-48, wherein the isolating comprising leukapheresis.

50. The method of any one of claims 46-49, wherein the isolating is performed on blood from the donor.

51. The method of any one of claims 46-50, wherein the donor is a human individual.

52. The method of any one of claims 46-51, further comprising obtaining blood from the donor.

53. The method of any one of claims 1-52, wherein the method further comprises performing a step of γδ T cell transduction.

54. The method of claims 53, wherein the culturing step is split by a step of transducing the cells in culture to obtain transduced γδ T cells.

55. The method of claim 54, wherein the culturing step comprises a first culturing step of about 0 to about 4 days, with the transducing performed on about day 4.

56. The method of claim 55, wherein the culturing step comprises a second culturing step of about 0 to about 10 days.

57. The method of any one of claims 53-56, wherein the transducing comprises introducing one or more transgenes.

58. The method of claim 57, wherein the one or more transgenes comprises a CAR.

59. The method of claim 58, wherein the CAR specifically recognizes globohexaosylceramide (GloboH), AKR1C3, SSEA-4, or TROP-2.

60. The method of any one of claims 1-59, further comprising cryopreserving the produced population of cells comprising γδ T cells following the harvesting step.

61. The method of any one of claims 1-60, wherein the method is a feeder cell-free method for producing the population of cells comprising γδ T cells.

62. The method of any one of claims 1-61, wherein the method does not comprise the use of an antibody in culturing the pool of mixed immune cells, or the derivative thereof, after αβ T cell depletion.

63. The method of any one of claims 1-62, wherein the method does not comprise the use of an aminobisphosphonate in culturing the pool of mixed immune cells, or the derivative thereof.

64. The method of claim 63, wherein the aminobisphosphonate is zoledronate or pamidronate, or a mixture thereof.

65. A population of cells comprising γδ T cells produced using a method of any one of claims 1-64.

66. The population of claim 60, wherein the population comprises at least about 1×10⁸ γδ T cells.

67. The population of claim 60 or 61, wherein the population comprises one or more transgenes.

68. The population of claim 62, wherein the one or more transgenes comprises a CAR.

69. A method of treating a disease in an individual, the method comprising:
obtaining an isolated pool of mixed immune cells depleted of αβ T cells, or a derivative thereof, from a donor individual;
producing a population of cells comprising γδ T cells using a method of any one of claims 1-64; and
administering the population of cells comprising γδ T cells to the individual.

70. The method of claim 69, wherein the disease is a cancer.

71. The method of claim 69 or 70, wherein the donor individual is not the individual.

72. The method of claim 69 or 70, wherein the donor individual is the individual.
